(19) United States
(12) Patent Application Publication
(10) Pub. No.: US 2003/0170674 A1
(43) Pub. Date: Sep. 11, 2003

(54) Nitric oxide synthase gene diagnostic polymorphisms

(75) Inventor: David W. Moskowitz, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: DZ Genes, LLC

(21) Appl. No.: 10/268,311

(22) Filed: Oct. 10, 2002

Related U.S. Application Data
(63) Continuation of application No. 09/769,207, filed on Jan. 24, 2001, now abandoned.
(60) Provisional application No. 60/220,662, filed on Jul. 25, 2000.
(60) Provisional application No. 60/177,775, filed on Jan. 24, 2000.

(51) Int. Cl. C12Q 1/68; C07H 21/04

(52) U.S. Cl. 435/6; 536/23.2

(57) ABSTRACT

Disclosed is a method for determining a genetic predisposition to hypertension, end stage renal disease due to hypertension, non-insulin dependent diabetes mellitus, end stage renal disease due to non-insulin dependent diabetes mellitus, breast cancer, lung cancer or prostate cancer by detecting the presence or absence of single nucleotide polymorphisms in the nitric oxide synthase gene. Also disclosed are kits for detecting the presence or absence of the single nucleotide polymorphisms, methods for the treatment and/or prophylaxis of diseases, conditions, or disorders associated with the single nucleotide polymorphisms.

### CROSS REFERENCE TO RELATED APPLICATIONS

This application claims the benefit of U.S. provisional application serial No. 60/177,775, filed Jan. 24, 2000 and U.S. provisional application serial No. 60/220,662 filed Jul. 25, 2000 both of which are herein incorporated by reference in their entirety.

### BACKGROUND

This invention relates to detection of an individual's genetic predisposition for a disease, condition or disorder based on the presence or absence of single nucleotide polymorphisms (SNPs).

During the course of evolution, spontaneous mutations appear in the genomes of organisms. It has been estimated that variations in genomic DNA sequences are created continuously at a rate of about 100 new single base changes per individual (Kondrashow, J. Theor. Biol., 175:583-594, 1995; Crow, Exp. Clin. Immunogenet., 12:121-128, 1995) These changes in the progenitor nucleotide sequences may confer an evolutionary advantage, in which case the frequency of the mutation will likely increase, an evolutionary disadvantage in which case the frequency of the mutation is likely to decrease, or the mutation will be neutral. In certain cases the mutation may be lethal in which case the mutation is not passed on to the next generation and so is quickly eliminated from the population. In many cases, an equilibrium is established between the progenitor and mutant sequences so that both are present in the population. The presence of both forms of the sequence results in genetic variation or polymorphism. Over time, a significant number of mutations can accumulate within a population such that considerable polymorphism can exist between individuals within the population.

Numerous types of polymorphism are known to exist. Polymorphisms can be created when DNA sequences are either inserted or deleted from the genome, for example, by viral insertion. Another source of sequence variation can be caused by the presence of repeated sequences in the genome variously termed short tandem repeats (STR), variable number tandem repeats (VNTR), short sequence repeats (SSR) or microsatellites. These repeats can be dinucleotide, trinucleotide, tetranucleotide or pentanucleotide repeats. Polymorphism results from variation in the number of repeated sequences found at a particular locus.

By far the most common source of variation in the genome are single nucleotide polymorphisms or SNPs. SNPs account for approximately 90% of human DNA polymorphism (Collins et al., Genome Res., 8:1229-1231, 1998). SNPs are single base pair positions in genomic DNA at which different sequence alternatives (alleles) exist in a population. Several definitions of SNPs exist in the literature (Brooks, Gene, 234:177-186, 1999). As used herein, the term “single nucleotide polymorphism” or “SNP” includes all single base variants and so includes nucleotide insertions and deletions in addition to single nucleotide substitutions(e.g. A→G). Nucleotide substitutions are of two types. A transition is the replacement of one purine by another purine or one pyrimidine by another pyrimidine. A transversion is the replacement of a purine for a pyrimidine or vice versa.

The typical frequency at which SNPs are observed is about 1 per 1000 base pairs (Li and Sadler, Genetics, 129:513-523, 1991; Wang et al., Science, 280:1077-1082, 1998; Harding et al., Am. J. Human Genet., 60:772-789, 1997; Taillon-Miller et al., Genome Res., 8:748-754, 1998) The frequency of SNPs varies with the type and location of the change. In base substitutions, two-thirds of the substitutions involve the C [symbol] T (G [symbol] A) type. This variation in frequency is thought to be related to 5-methylcytosine deamination reactions that occur frequently, particularly at CpG dinucleotides. In regard to location, SNPs occur at a much higher frequency in non-coding regions than they do in coding regions.

SNPs can be associated with disease conditions in humans or animals. The association can be direct as in the case of genetic diseases where the alteration in the genetic code caused by the SNP directly results in the disease condition. Examples of diseases in which single nucleotide polymorphisms result in disease conditions are sickle cell anemia and cystic fibrosis. The association can also be indirect where the SNP does not directly cause the disease but alters the physiological environment such that there is an increased likelihood that the patient will develop the disease. SNPs can also be associated with disease conditions, but play no direct or indirect role in causing the disease. In this case, the SNP is located close to the defective gene, usually within 5 centimorgans, such that there is a strong association between the presence of the SNP and the disease state. Because of the high frequency of SNPs within the genome, there is a greater probability that a SNP will be linked to a genetic locus of interest than other types of genetic markers.

Disease associated SNPs can occur in coding and non-coding regions of the genome. When located in a coding region, the presence of the SNP can result in the production of a protein that is non-functional or has decreased function. More frequently, SNPs occur in non-coding regions. If the SNP occurs in a regulatory region, it may affect expression of the protein. For example, the presence of a SNP in a promoter region, may cause decreased expression of a protein. If the protein is involved in protecting the body against development of a pathological condition, this decreased expression can make the individual more susceptible to the condition.

Numerous methods exist for the detection of SNPs within a nucleotide sequence. A review of many of these methods can be found in Landegren et al., Genome Res., 8:769-776, 1998. SNPs can be detected by restriction fragment length polymorphism (RFLP)(U.S. Pat. Nos. 5,324,631, 5,645,995). RFLP analysis of the SNPs, however, is limited to cases where the SNP either creates or destroys a restriction enzyme cleavage site. SNPs can also be detected by direct sequencing of the nucleotide sequence of interest. Numerous assays based on hybridization have also been developed to detect SNPs. In addition, mismatch distinction by polymerases and ligases have also been used to detect SNPs.

There is growing recognition that SNPs can provide a powerful tool for the detection of individuals whose genetic make-up alters their susceptibility to certain diseases. There are four primary reasons why SNPs are especially suited for the identification of genotypes that influence an individual's predisposition to a disease condition. First, SNPs are by far the most prevalent type of polymorphism present in the genome and so are likely to be present in or near any locus of interest. Second, SNPs located in genes can be expected to directly affect protein structure or expression levels and so may serve not only as markers, but as candidates for gene therapy treatments to treat or prevent a disease. Third, SNPs show greater genetic stability than repeated sequences and so are less likely to undergo changes which would complicate diagnosis. Fourth, the increasing efficiency of methods of detection of SNPs make them especially suitable for high throughput typing systems necessary to screen large populations.

One disease for which the discovery of markers to detect increased genetic susceptibility is critically needed is end-stage renal disease. End-stage renal disease (ESRD) is defined as the condition when life becomes impossible without replacement of renal functions either by kidney dialysis or kidney transplantation. Hypertension (HTN) and non-insulin dependent diabetes (NIDDM) are the leading causes of end-stage renal disease (ESRD) nationally (United States Renal Data System, Table IV-3, p. 49, 1994). There is currently an epidemic of ESRD, due mainly to the aging of the American population. The ESRD epidemic is of special concern among African Americans where the incidence of ESRD is four- to six-fold higher than for Caucasians (Brancati et al., J. Am. Med. Assoc., 268:3079-3084, 1992), but where treatment of hypertension, a causative factor in ESRD, is less effective (Walker et al., J. Am. Med. Assoc., 268:3085-3091, 1992).

There are over 200,000 patients with ESRD receiving renal replacement therapy (dialysis or renal transplantation), with an annual cost of $13 billion. These numbers will certainly increase as the population of the nation continues to age. Since 1980, when complete data became available for the first time, most new cases of ESRD have been ascribed to NIDDM or hypertension. The incidence of ESRD due to NIDDM or hypertension is still increasing, suggesting that the U.S. is in the early phase of an epidemic of ESRD. Preventing ESRD would save at least $30,000 per patient per year in dialysis costs alone, as well as enhance the patient's quality of life and ability to work. It is clearly the ideal method of cost-containment for renal disease. Without effective prevention of ESRD, the nation will instead be forced to adopt less humane methods of cost-containment, such as denial of access (gate-keeping), or rely upon unrealistic expectations about patient reimbursement rates, etc.

Nitric Oxide (NO) has been recognized as a potential factor in the progression of chronic renal failure (Aiello et al., Kidney Intl. Suppl., 65:S63-S67, 1998). Nitric oxide, a readily diffusible gas identical to endothelium-derived relaxing factor (EDRF), is synthesized by nitric oxide synthase (NOS). Three isoforms of NOS exist: inducible NOS (INOS; NOS1), neuronal NOS(NNOS; NOS2), and endothelial constitutive NOS (ecNOS, NOS3).

Nitric oxide (NO) has been strongly implicated in apoptosis of endothelial (Bonfoco et al., Proc. Natl. Acad. Sci. USA, 92:7162-7166, 1995) and vascular smooth muscle cells (Nishio et al., Biochem. Biophys. Res. Commun., 221:163-168, 1996). Nitric oxide, which is vasodilatory, antagonizes the vasoconstrictive effects of angiotensin II and endothelins. Since angiotensin II promotes renal injury, nitric oxide may protect against renal injury from systemic disease such as hypertension and non-insulin dependent diabetes mellitus (NIDDM; Bataineh and Raij, Kidney Int., Suppl., 68:S140S19, 1998). Nitric oxide has also been implicated in the progression of renal disease in rats (Brooks and Contino, Pharmacology, 56:257-261, 1998) and humans (Noris and Remuzzi, Contrib. Nephrol. 119:8-15, 1996; Kone, Am. J. Kidney Dis., 30:311-333, 1997; Aiello et al., Kidney Int., Suppl., 65:S63-S67, 1998; Raij, Hypertension, 31:189-193, 1998). The nitric oxide synthase genes are recognized candidate genes for hypertension, renal failure, and cardiovascular disease in general (Soubrier, Hypertension, 31:189-193, 1998)

NO can directly oxidize (and activate) thiol-containing proteins such as NF-KB (nuclear factor-kappaB) and AP-1 (Activator Protein 1) (Stamler, Cell, 78:931-936, 1994). NO can either promote apoptosis or prevent it. Above a threshold concentration, NO seems to stimulate apoptosis (Bonfoco et al., Proc. Natl. Acad. Sci. USA, 92:7162-7166, 1995; Stamler, Cell, 78:931-936, 1994).

The highest amount of NO is made by inducible NO synthase (INOS, NOS II), which is fully active at the prevailing intracellular calcium concentration (Caᵢ ˜100 nM), and once induced, remains active for days producing nanomolar amounts of NO (Yu et al., Proc. Natl. Acad. Sci. USA, 91:1691-1695, 1994). The cis regulatory sequences for iNOS are not fully known. However, a region of 1798 nucleotides (nt) immediately upstream (5′) of the gene has been sequenced. Additional regulatory regions far upstream have been found in the human iNOS gene (de Vera ME et al., Proc. Natl. Acad. Sci. USA, 93:1054-1059, 1996). Increased inducibility of iNOS would have conferred an important selection advantage, since iNOS is thought to be the major mechanism for immune cell-mediated killing of infectious agents such as parasites (e.g. malaria), bacteria, and viruses.

An additional source of renal NO is endothelial constitutive NOS (ecNOS, NOS III). ecNOS requires an elevation of intracellular calcium (Caᵢ) to be active, since it must bind calmodulin for activity. ecNOS, which produces picomolar amounts of NO, may seem an unlikely source of large amounts of NO, but it is specifically activated by shear stress (Awolesi et al., Surgery, 116:439-445, 1994), and may be involved in arterial remodeling. Like adenosine and endothelin-1, ecNOS may therefore account for the clinical observation that the rate of progression of chronic renal failure (CRF) is proportional to the degree of hypertension. Single nucleotide variations in the 5′ promoter region (1600 nt) of ecNOS might thus allow for increased induction.

L-arginine, a substrate for nitric oxide production, is an essential amino acid that can be given orally. Two studies in rats with subtotal nephrectomy (Reyes et al., Am. J. Kidney Dis., 20:168-176, 1992; Ashab et al., Kidney Intl., 47:1515-1521, 1995) have shown improvement of renal function with oral administration of L-arginine, suggesting that low levels of NO may play a role in the development of ESRD. Concentrations of 1.25 to 10 grams/liter of L-arginine were used in the rat studies resulting in a dose of approximately 1.25 to 10 grams/kg body weight/day. In a recent human trial, however, administration of only 0.2 gram/kg body weight/day of L-arginine had no demonstrable effect (De Nicola et al., Kidney Intl., 56:674-684, 1999).

In the remnant kidney model of chronic renal failure in rats, activity of ecNOS remains unchanged whereas the activity of iNOS decreases markedly (Aiello et al., Kidney Intl. 52:171-181, 1997). A deficiency of nitric oxide, especially due to the ecNOS isoform which normally remains unchanged after renal injury, may predispose patients with underlying systemic disease to end-stage renal disease (ESRD) (Huang, Am. J. Cardiol., 82:57S-59S, 1998).

A number of polymorphisms have been reported in the sequence of the ecNOS gene, some of which have also been reported to be associated with variations in plasma levels of NO (Wang et al., Arterioscler. Thromb. Vasc. Biol., 17:3147-3153, 1997; Tsukada et al., Biochem Biophys. Res. Commun., 245:190-193, 1998)

Nakayama et al. (Hum. Hered., 45:301-302, 1995; Clin. Genet., 51:26-30, 1997), have reported the presence of highly polymorphic (CA)n repeats in intron 13 of the ecNOS promoter. Bonnardeaux et al. (Circulation, 91:96-102, 1995), reported the presence of two biallelic markers in intron 18 that were not linked to essential hypertension.

Two forms of a 27 base pair repeat in intron 4 have been reported; a larger allele, with 5 tandem repeats, and a smaller allele, with only 4 repeats (third repeat missing). The rare, smaller allele has been associated with coronary artery disease in smokers, but not in patients who had never smoked (Wang et al., Nat. Med., 2:41-45, 1996; Ichihara et al., Am. J. Cardiol., 81:83-86, 1998). The smaller allele has also been associated with essential hypertension (Uwabo et al., Am. J. Hypertens., 11:125-128, 1998). An additional association was also observed in Turkish patients with deep vein thrombosis and strokes (Akar et al., Thromb. Res., 94:63064, 1999). Several studies, however, failed to confirm any association of the intron 4 polymorphism with cardiovascular disease (Yahashi et al., Blood Coagul. Fibrinolysis, 9:405-409, 1998), essential hypertension (Bonnardeaux et al., Circulation, 91:96-102, 1995), or of the ecNOS gene with myocardial infarction (Poirier et al., Eur. J. Clin. Invest., 29:284-290, 1999)

A missense Glutamate 298 to Aspartate variant (E298D) in exon 7 has been associated with coronary spasm in Japanese patients (Yoshimura et al., Hum. Genet., 103:65-69, 1998) as well as enhanced vasoconstriction by phenylephrine (Philip et al., Circulation, 99:3096-3098, 1999). Despite observed associations with coronary spasm (Yoshimura et al., Hum. Genet., 103:65-69, 1998) and preeclampsia, there was no linkage of ecNOS with migraine headaches, which are also thought to involve arterial spasm (Griffiths et al., Neurology, 49:614-617, 1997). The E298D polymorphism was also associated with essential hypertension in some studies (Miyamoto et al., Hypertension, 32:3-8, 1998; Yasujima et al., Rinsho Byori, 46:1199-1204, 1998) but no association was seen in a larger study (Kato et al., Hypertension, 33:933-936, 1999), nor was the E298D polymorphism associated with a measure of aortic stiffness, a consequence of hypertension (Lacolley et al., J. Hypertens., 16:31-35, 1998). The findings regarding a possible association between the E298D polymorphism and myocardial infarction have been mixed, with an association found in some studies (Hibi et al., Hypertension, 32:521-526, 1998; Shimasaki et al., J. Am. Coll. Cardiol., 31:1506-1510, 1998; Hingorani et al., Circulation, 100:1515-1520, 1999), but not others (Cai et al., J. Mol. Med. 77:511-514, 1999; Liyou et al., Clin. Genet., 54:528-529, 1998). Likewise, there have been mixed findings regarding assoications between the E298D polymorphism and cerebrovascular disease in Caucasians with Markus et al. (Stroke, 29:1908-1911, 1998) and MacLeod et al. (Neurology, 53:418-420, 1999) finding no association while Elbaz et al. (Stroke 31:1634-1639, 2000) found an association of the E298D mutation with brain infarction.

Brscic et al. (Am. Heart J. 139:979-984, 2000) studied various genetic polymorphisms in angiotensin I converting enzyme, angiotensin II type I receptor, apolipoprotein E, endothelial constitutive nitric oxide synthase, and platelet glycoprotein IIIa and their possible association with myocardial infarction. A significant assoication with myocardial infarction was found only with polymorhisms in the apolipoprotein gene.

Neugebauer et al. (Diabetes 49:500-503, 2000) investigated ecNOS tandem repeat polymorphism and found no association with hypertension or diabetic retinopathy. Similar results were obtained by Warpeha et al. (Eye 13:174-178, 1999). Likewise, Smyth et al. (Rheumatology 38:1094-1098, 1999) found no association between allele frequencies in the eNOS gene and Raynaud's phenomenon. Conflicting reports have been published regarding the possible role of the eNOS gene in preeclampsia. Lade et al. (Hypertens. Pregnancy 18:81-93, 1999) examined two microsatellite markers (D7S483 and D7S505) in proximity of the eNOS gene and found no association with preeclampsia in contrast to the earlier findings of Arngrimsson et al. (Am. J. Hum. Genet. 61:354-362, 1997)

Polymorphisms in the promoter of ecNOS have also been described. A mutation at position −786 of T to C has been reported which was associated with coronary spasm (Nakayama et al., Circulation, 99:2864-2870, 1999). Also seen were an A-to-G mutation at position −922, and a T-to-A mutation at position −1468, which were linked to the T-786→C mutation. However, in a luciferase construct, only the T-786→C mutation resulted in a significant reduction in ecNOS gene promoter activity (Id.; Yoshimura et al., J. Investig. Med. 48:367-374, 2000). Position −786 corresponds to position +2684 in the promoter sequence contained in GenBank as accession number AF032908 (SEQ ID NO: 1).

Zanchi et al. (Kidney Intl. 57:405-413, 2000) examined the T-786→C substitution in the promoter regions and an a-deletion/b-insertion in intron 4 of the ecNOS gene. They reported that both mutations were associated with a risk of advanced nephropathy in type 1 (insulin dependent) diabetes.

A MspI restriction fragment length polymorphism (RFLP) has been reported in an Australian Caucasian population (Sim et al., Mol. Genet. Metab., 65:562, 1998). The T to C mutation at position −781 (AF032908 position 2692) was not shown to be associated with any human disease nor to be functional when cloned upstream of a luciferase reporter gene in HepG2 cells.

An additional C to T mutation has also been reported at position −690 (Nishio et al., Biochem. Biophys. Res. Commun., 221:163-168, 1996), corresponding to position +2783 in the promoter sequence AF032908 (Tunny et al., Clin. Exp. Pharmacol Physiol., 25:26-29, 1998).

An ideal approach to prevention of ESRD would be the identification of any genes that predispose an individual to ESRD early enough to be able to counteract this predisposition. Knowledge of ESRD-predisposing genes is essential for truly effective delay, or, ideally, prevention of ESRD.

### SUMMARY

The present inventor has discovered novel associations of single nucleotide polymorphisms (SNPs) within the nucleic acid sequence encoding endothelial constitutive nitric oxide synthase and associated regulatory regions with various disease. As such, these polymorphisms provide a method for diagnosing a genetic predisposition for the development of a disease in individuals. Information obtained from the detection of SNPs associated with an individuals genetic predisposition to a disease is of great value in the treatment and prevention of the disease.

Accordingly, one aspect of the present invention provides a method for diagnosing a genetic predisposition for a disease, condition or disorder in a subject comprising, obtaining a biological sample containing nucleic acid from said subject; and analyzing said nucleic acid to detect the presence or absence of a single nucleotide polymorphism in SEQ ID NO: 1 or the complement thereof, wherein said single nucleotide polymorphism is associated with a genetic predisposition for a disease condition or disorder selected from the group consisting of hypertension, end stage renal disease due to hypertension, non-insulin dependent diabetes mellitus, end stage renal disease due to non-insulin dependent diabetes mellitus, breast cancer, lung cancer, and prostate cancer.

Another aspect of the present invention provides an isolated polynucleotide comprising at least 10 contiguous nucleotides of SEQ ID NO: 1 or the complement thereof, and containing at least one single nucleotide polymorphism associated with a disease, condition or disorder selected from the group consisting of hypertension, end stage renal disease due to hypertension, non-insulin dependent diabetes mellitus, end stage renal disease due to non-insulin dependent diabetes mellitus, breast cancer, lung cancer, and prostate cancer.

Yet another aspect of the invention provides a kit comprising at least one isolated polynucleotide of at least 10 continuous nucleotides of SEQ ID NO: 1 or the complement thereof, and containing at least one single nucleotide polymorphism associated with a disease, condition or disorder selected from the group consisting of hypertension, end stage renal disease due to hypertension, non-insulin dependent diabetes mellitus, end stage renal disease due to non-insulin dependent diabetes mellitus, breast cancer, lung cancer, and prostate cancer; and instructions for using said polynucleotide for detecting the presence or absence of said at least one single nucleotide polymorphism in said nucleic acid.

Yet another aspect of the invention provides a kit comprising at least one polynucleotide of at least 10 contiguous nucleotides of SEQ ID NO: 1 or the complement thereof, wherein the 3′ end of said polynucleotide is immediately 5′ to a single nucleotide polymorphism site associated with a genetic predisposition to disease condition, or disorder selected from the group consisting of hypertension, end stage renal disease due to hypertension, non-insulin dependent diabetes mellitus, end stage renal disease due to non-insulin dependent diabetes mellitus, breast cancer, lung cancer, and prostate cancer; and instructions for using said polynucleotide for detecting the presence or absence of said single nucleotide polymorphism in a biological sample containing nucleic acid.

Still another aspect of the invention provides a method for treatment or prophylaxis in a subject comprising, obtaining a sample of biological material containing nucleic acid from a subject; analyzing said nucleic acid to detect the presence or absence of at least one single nucleotide polymorphism in SEQ ID NO: 1 or the complement thereof, associated with a disease, condition or disorder selected from the group consisting of hypertension, end stage renal disease due to hypertension, non-insulin dependent diabetes mellitus, end stage renal disease due to non-insulin dependent diabetes mellitus, breast cancer, lung cancer, and prostate cancer; and treating said subject for said disease, condition or disorder.

Further scope of the applicability of the present invention will become apparent from the detailed description provided below. It should be understood, however, that the following detailed description and examples, while indicating preferred embodiments of the invention, are given by way of illustration only, since various changes and modifications within the spirit and scope of the invention will become apparent to those skilled in the art from the following detailed description.

### Definitions

bp=base pair

kb=kilobase; 1000 base pairs

ESRD=end-stage renal disease

HTN=hypertension

NIDDM=noninsulin-dependent diabetes mellitus

CRF=chronic renal failure

T-GF=tubulo-glomerular feedback

CRG=compensatory renal growth

MODY=maturity-onset diabetes of the young

RFLP=restriction fragment length polymorphism

MASDA=multiplexed allele-specific diagnostic assay

MADGE=microtiter array diagonal gel electrophoresis

OLA=oligonucleotide ligation assay

DOL=dye-labeled oligonucleotide ligation assay

SNP=single nucleotide polymorphism

PCR=polymerase chain reaction

As used herein “polynucleotide” and “oligonucleotide” are used interchangeably and refer to a polymeric (2 or more monomers) form of nucleotides of any length, either ribonucleotides or deoxyribonucleotides. Although nucleotides are usually joined by phosphodiester linkages, the term also includes polymeric nucleotides containing neutral amide backbone linkages composed of aminoethyl glycine units. This term refers only to the primary structure of the molecule. Thus, this term includes double- and single-stranded DNA and RNA. It also includes known types of modifications, for example, labels, methylation, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.), those containing pendant moieties, such as, for example, proteins (including for e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide. Polynucleotides include both sense and antisense strands.

“Sequence” means the linear order in which monomers occur in a polymer, for example, the order of amino acids in a polypeptide or the order of nucleotides in a polynucleotide.

“Polymorphism” refers to a set of genetic variants at a particular genetic locus among individuals in a population.

“Gene therapy” means the introduction of a functional gene or genes from some source by any suitable method into a living cell to correct for a genetic defect.

“Reference sequence” means SEQ ID NO: 1.

“Genetic variant” or “variant” means a specific genetic variant which is present at a particular genetic locus in at least one individual in a population and that differs from a reference sequence.

As used herein the terms “patient” and “subject” are not limited to human beings, but are intended to include all vertebrate animals in addition to human beings.

As used herein, the terms “genetic predisposition”, “genetic susceptibility” and “susceptibility” all refer to the likelihood that an individual subject will develop a particular disease, condition or disorder. For example, a subject with an increased susceptibility or predisposition will be more likely that average to develop a disease, while a subject with a decreased predisposition will be less likely than average to develop the disease. A genetic variant is associated with an altered susceptibility or predisposition if the calculated odds ratio is 1.5 or greater. Alternatively, a genetic variant is associated with an altered susceptibility or predisposition if the allele frequency of the genetic variant in a population or subpopulation with a disease, condition or disorder varies from its allele frequency in the population without the disease, condition or disorder (control population) or a reference sequence (wild type) by at least 1%, preferably by at least 2%, more preferably by at least 4% and more preferably still by at least 8%.

### DETAILED DESCRIPTION

All publications, patents, patent applications databases and other references cited in this application are herein incorporated by reference in their entirety as if each individual publication, patent, patent application, database or other reference was specifically and individually indicated to be incorporated by reference.

Novel Polymorphisms

The human endothelial constitutive nitric oxide snythase (ecNOS,NOS3) gene promoter region resides on chromosome 7. The sequence of the ecNOS promoter has been published (GenBank accession #AF032908)(SEQ ID NO: 1). This sequence includes the ecNOS regulatory regions and the first 31 amino acids of the protein coding region (SEQ ID NO: 2). The present application provides single nucleotide polymorphisms (SNPs) within the ecNOS promoter region and preferably at positions 2548, 2684, 2575, 1272, 2841, 2843 and 3556. Positions of the single nucleotide polymorphisms are given according to the numbering scheme in GenBank accession No. AF032908. Thus, all nucleotide positions are denoted by positive numbers.

Preparation of Samples

The presence of genetic variants in the reference sequence is determined by screening nucleic acid sequences from a population of individuals for such variants. The population is preferably comprised of some individuals with the disease of interest, so that any genetic variants that are found can be correlated with the disease. The population is also preferably comprised of some individuals that have known risk for the disease, such as individuals with hypertension, NIDDM, or CRF. The population should preferably be large enough to have a reasonable chance of finding individuals with the sought-after genetic variant. As the size of the population increases, the ability to find significant correlations between a particular genetic variant and susceptibility to the disease of interest also increases. Preferably, the population should have 10 or more individuals.

The nucleic acid sequence can be DNA or RNA. For the assay of genomic DNA, virtually any biological sample containing genomic DNA (e.g. not pure red blood cells) can be used. For example, and without limitation, genomic DNA can be conveniently obtained from whole blood, semen, saliva, tears, urine, fecal material, sweat, buccal cells, skin or hair. For assays using cDNA or mRNA, the target nucleic acid can be obtained from cells or tissues that express the target sequence. One preferred source and quantity of DNA is 10 to 30 ml of anticoagulated whole blood, since enough DNA can be extracted from leukocytes in such a sample to perform many repetitions of the analysis contemplated herein.

Many of the methods described herein require the amplification of DNA from target samples. This can be accomplished by any method known in the art but preferably by the polymerase chain reaction (PCR). Optimization of conditions for conducting PCR must be determined for each reaction and can be accomplished without undue experimentation by one of ordinary skill in the art. In general, methods for conducting PCR can be found in U.S. Pat. Nos. 4,965,188, 4,800,159, 4,683,202, and 4,683,195; Ausbel et al., eds., Short Protocols in Molecular Biology, 3^{rd} ed., Wiley, 1995; and Innis et al., eds., PCR Protocols, Academic Press, 1990.

Other amplification methods include the ligase chain reaction (LCR) (see, Wu and Wallace, Genomics, 4:560-569, 1989; Landegren et al., Science, 241:1077-1080, 1988), transcription amplification (Kwoh et al., Proc. Natl. Acad. Sci. USA, 86:1173-1177, 1989), self-sustained sequence replication (Guatelli et al., Proc. Natl. Acad. Sci. USA, 87:1874-1878, 1990), and nucleic acid based sequence amplification (NASBA). The latter two amplification methods involve isothermal reactions based on isothermal transcription, which produces both single stranded RNA (ssRNA) and double stranded DNA (dsDNA) as the amplification products in a ratio of about 30 or 100 to 1, respectively.

Detection of Polymorphisms

Detection of Unknown Polymorphisms

Two types of detection are contemplated within the present invention. The first type involves detection of unknown SNPs by comparing nucleotide target sequences from individuals in order to detect sites of polymorphism. If the most common sequence of the target nucleotide sequence is not known, it can be determined by analyzing individual humans, animals or plants with the greatest diversity possible. Additionally the frequency of sequences found in subpopulations characterized by such factors as geography or gender can be determined.

The presence of genetic variants and in particular SNPs is determined by screening the DNA and/or RNA of a population of individuals for such variants. If it is desired to detect variants associated with a particular disease or pathology, the population is preferably comprised of some individuals with the disease or pathology, so that any genetic variants that are found can be correlated with the disease of interest. It is also preferable that the population be composed of individuals with known risk factors for the disease. The populations should preferably be large enough to have a reasonable chance to find correlations between a particular genetic variant and susceptibility to the disease of interest. In one embodiment, the population preferably has at least 10 individuals, in another embodiment, the population preferably has 100 individuals or more. In one embodiment, the population is preferably comprised of individuals that have known risk factors for ESRD, breast cancer, lung cancer and prostate cancer.,

Determination of unknown genetic variants, and in particular SNPS, within a particular nucleotide sequence among a population may be determined by any method known in the art, for example and without limitation, direct sequencing, restriction length fragment polymorphism (RFLP), single-strand conformational analysis (SSCA), denaturing gradient gel electrophoresis (DGGE), heteroduplex analysis (HET), chemical cleavage analysis (CCM) and ribonuclease cleavage.

Methods for direct sequencing of nucleotide sequences are well known to those skilled in the art and can be found for example in Ausubel et al., eds., Short Protocols in Molecular Biology, 3^{rd} ed., Wiley, 1995 and Sambrook et al., Molecular Cloning, 2^{nd} ed., Chap. 13, Cold Spring Harbor Laboratory Press, 1989. Sequencing can be carried out by any suitable method, for example, dideoxy sequencing (Sanger et al., Proc. Natl. Acad. Sci. USA, 74:5463-5467, 1977), chemical sequencing (Maxam and Gilbert, Proc. Natl. Acad. Sci. USA, 74:560-564, 1977) or variations thereof. Direct sequencing has the advantage of determining variation in any base pair of a particular sequence.

In one embodiment, direct sequencing is accomplished by pyrosequencing. In pyrosequencing a sequencing primer is hybridized with a DNA template and incubated with the enzymes DNA polymerase, ATP sulfurylase, luciferase and apyrase, and the substrates, adenosine 5′ phosphosulfate (APS) and luciferin. The first of four deoxynucleotide triphosphates (dNTP) is added to the reaction and incorporated into the DNA primer strand if it is complementary to the base in the template. Each dNTP incorporation is accompanied by release of pyrophosphate (PPi) in an quantity equimolar to the amount of incorporated nucleotide. ATP sylfurylase then quantitatively converts the PPi to ATP in the presence of adenosine 5′ phosphosulfate. The ATP produced drives the luciferase mediated conversion of luciferin to oxyluciferin which generates visible light in amounts proportional to the amount of ATP. The amount of light produced is measured and is proportional to the number of nucleotides incorporated. The reaction is then repeated for each of the remaining dNTPs. For DATP, alfa-thio triphosphate (dATPαS) is used since it is efficiently utilized by DNA polymerase but not by luciferase. Methods for using pyrosequencing to detect SNPs are known in the art and can be found. for example, in Alderborn et al., Genome Res. 10:1249-1258, 2000; Ahmadian et al., Anal. Biochem. 10:103-110, 2000; and Nordstrom et al., Biotechnol. Appl. Biochem. 31:107-112, 2000.

RFLP analysis (see, e.g. U.S. Pat. No. 5,324,631 and 5,645,995) is useful for detecting the presence of genetic variants at a locus in a population when the variants differ in the size of a probed restriction fragment within the locus, such that the difference between the variants can be visualized by electrophoresis. Such differences will occur when a variant creates or eliminates a restriction site within the probed fragment. RFLP analysis is also useful for detecting a large insertion or deletion within the probed fragment. Thus, RFLP analysis is useful for detecting, e.g., an Alu sequence insertion or deletion in a probed DNA segment.

Single-strand conformational polymorphisms (SSCPs) can be detected in <220 bp PCR amplicons with high sensitivity (Orita et al, Proc. Natl. Acad. Sci. USA, 86:2766-2770, 1989; Warren et al., In: Current Protocols in Human Genetics, Dracopoli et al., eds, Wiley, 1994, 7.4.1-7.4.6.). Double strands are first heat-denatured. The single strands are then subjected to polyacrylamide gel electrophoresis under non-denaturing conditions at constant temperature (i.e. low voltage and long run times) at two different temperatures, typically 4-10° C. and 23° C. (room temperature). At low temperatures (4-10° C.), the secondary structure of short single strands (degree of intrachain hairpin formation) is sensitive to even single nucleotide changes, and can be detected as a large change in electrophoretic mobility. The method is empirical, but highly reproducible, suggesting the existence of a very limited number of folding pathways for short DNA strands at the critical temperature. Polymorphisms appear as new banding patterns when the gel is stained.

Denaturing gradient gel electrophoresis (DGGE) can detect single base mutations based on differences in migration between homo- and heteroduplexes (Myers et al., Nature, 313:495-498, 1985). The DNA sample to be tested is hybridized to a labeled wild type probe. The duplexes formed are then subjected to electrophoresis through a polyacrylamide gel that contains a gradient of DNA denaturant parallel to the direction of electrophoresis. Heteroduplexes formed due to single base variations are detected on the basis of differences in migration between the heteroduplexes and the homoduplexes formed.

In heteroduplex analysis (HET)(Keen et al., Trends Genet. 7:5, 1991), genomic DNA is amplified by the polymerase chain reaction followed by an additional denaturing step which increases the chance of heteroduplex formation in heterozygous individuals. The PCR products are then separated on Hydrolink gels where the presence of the heteroduplex is observed as an additional band.

Chemical cleavage analysis (CCM)is based on the chemical reactivity of thymine (T) when mismatched with cytosine, guanine or thymine and the chemical reactivity of cytosine(C) when mismatched with thymine, adenine or cytosine (Cotton et al., Proc. Natl. Acad. Sci. USA, 85:4397-4401, 1988). Duplex DNA formed by hybridization of a wild type probe with the DNA to be examined, is treated with osmium tetroxide for T and C mismatches and hydroxylamine for C mismatches. T and C mismatched bases that have reacted with the hydroxylamine or osmium tetroxide are then cleaved with piperidine. The cleavage products are then analyzed by gel electrophoresis.

Ribonuclease cleavage involves enzymatic cleavage of RNA at a single base mismatch in an RNA:DNA hybrid (Myers et al., Science 230:1242-1246, 1985). A ³²P labeled RNA probe complementary to the wild type DNA is annealed to the test DNA and then treated with ribonuclease A. If a mismatch occurs, ribonuclease A will cleave the RNA probe and the location of the mismatch can then be determined by size analysis of the cleavage products following gel electrophoresis.

Detection of Known Polymorphisms

The second type of polymorphism detection involves determining which form of a known polymorphism is present in individuals for diagnostic or epidemiological purposes. In addition to the already discussed methods for detection of polymorphisms, several methods have been developed to detect known SNPs. Many of these assays have been reviewed by Landegren et al., Genome Res., 8:769-776, 1998 and will only be briefly reviewed here.

One type of assay has been termed an array hybridization assay, an example of which is the multiplexed allele-specific diagnostic assay (MASDA)(U.S. Pat. No. 5,834,181; Shuber et al., Hum. Molec. Genet., 6:337-347, 1997). In MASDA, samples from multiplex PCR are immobilized on a solid support. A single hybridization is conducted with a pool of labeled allele specific oligonucleotides (ASO). Any ASO that hybridizes to the samples are removed from the pool of ASOs. The support is then washed to remove unhybridized ASOs remaining in the pool. Labeled ASOs remaining on the support are detected and eluted from the support. The eluted ASOs are then sequenced to determine the mutation present.

Two assays depend on hybridization-based allele-discrimination during PCR. The TaqMan assay (U.S. Pat. No. 5,962,233; Livak et al., Nature Genet., 9:341-342, 1995) uses allele specific (ASO) probes with a donor dye on one end and an acceptor dye on the other end such that the dye pair interact via fluorescence resonance energy transfer (FRET). A target sequence is amplified by PCR modified to include the addition of the labeled ASO probe. The PCR conditions are adjusted so that a single nucleotide difference will effect binding of the probe. Due to the 5′ nuclease activity of the Taq polymerase enzyme, a perfectly complementary probe is cleaved during PCR while a probe with a single mismatched base is not cleaved. Cleavage of the probe dissociates the donor dye from the quenching acceptor dye, greatly increasing the donor fluorescence.

An alternative to the TaqMan assay is the molecular beacons assay (U.S. Pat. No. 5,925,517; Tyagi et al., Nature Biotech., 16:49-53, 1998). In the molecular beacons assay, the ASO probes contain complementary sequences flanking the target specific species so that a hairpin structure is formed. The loop of the hairpin is complimentary to the target sequence while each arm of the hairpin contains either donor or acceptor dyes. When not hybridized to a donor sequence, the hairpin structure brings the donor and acceptor dye close together thereby extinguishing the donor fluorescence. When hybridized to the specific target sequence, however, the donor and acceptor dyes are separated with an increase in fluorescence of up to 900 fold. Molecular beacons can be used in conjunction with amplification of the target sequence by PCR and provide a method for real time detection of the presence of target sequences or can be used after amplification.

High throughput screening for SNPs that affect restriction sites can be achieved by Microtiter Array Diagonal Gel Electrophoresis (MADGE)(Day and Humphries, Anal. Biochem., 222:389-395, 1994). In this assay, restriction fragment digested PCR products are loaded onto stackable horizontal gels with the wells arrayed in a microtiter format. During electrophoresis, the electric field is applied at an angle relative to the columns and rows of the wells allowing products from a large number of reactions to be resolved.

Additional assays for SNPs depend on mismatch distinction by polymerases and ligases. The polymerization step in PCR places high stringency requirements on correct base pairing of the 3′ end of the hybridizing primers. This has allowed the use of PCR for the rapid detection of single base changes in DNA by using specifically designed oligonucleotides in a method variously called PCR amplification of specific alleles (PASA)(Sommer et al., Mayo Clin. Proc., 64:1361-1372 1989; Sarker et al., Anal. Biochem. 1990), allele-specific amplification (ASA), allele-specific PCR, and amplification refractory mutation system (ARMS)(Newton et al., Nuc. Acids Res., 1989; Nichols et al., Genomics, 1989; Wu et al., Proc. Natl. Acad. Sci. USA, 1989). In these methods, an oligonucleotide primer is designed that perfectly matches one allele but mismatches the other allele at or near the 3′ end. This results in the preferential amplification of one allele over the other. By using three primers that produce two differently sized products, it can be determine whether an individual is homozygous or heterozygous for the mutation (Dutton and Sommer, BioTechniques, 11:700-702, 1991). In another method, termed bi-PASA, four primers are used; two outer primers that bind at different distances from the site of the SNP and two allele specific inner primers (Liu et al., Genome Res., 7:389-398, 1997). Each of the inner primers have a non-complementary 5′ end and form a mismatch near the 3′ end if the proper allele is not present. Using this system, zygosity is determined based on the size and number of PCR products produced.

The joining by DNA ligases of two oligonucleotides hybridized to a target DNA sequence is quite sensitive to mismatches close to the ligation site, especially at the 3′ end. This sensitivity has been utilized in the oligonucleotide ligation assay (OLA)(Landegren et al., Science, 241:1077-1080, 1988) and the ligase chain reaction (LCR; Barany, Proc. Natl. Acad. Sci. USA, 88:189-193, 1991). In OLA, the sequence surrounding the SNP is first amplified by PCR, whereas in LCR, genomic DNA can by used as a template.

In one method for mass screening for SNPs based on the OLA, amplified DNA templates are analyzed for their ability to serve as templates for ligation reactions between labeled oligonucleotide probes (Samotiaki et al., Genomics, 20:238-242, 1994). In this assay, two allele-specific probes labeled with either of two lanthamide labels (europium or terbium) compete for ligation to a third biotin labeled phosphorylated oligonucleotide and the signals from the allele specific oligonucleotides are compared by time-resolved fluorescence. After ligation, the oligonucleotides are collected on an avidin-coated 96-pin capture manifold. The collected oligonucleotides are then transferred to microtiter wells in which the europium and terbium ions are released. The fluorescence from the europium ions is determined for each well, followed by measurement of the terbium fluorescence.

In alternative gel-based OLA assays, numerous SNPs can be detected simultaneously using multiplex PCR and multiplex ligation (U.S. Pat. No. 5,830,711; Day et al., Genomics, 29:152-162, 1995; Grossman et al., Nuc. Acids Res., 22:4527-4534, 1994). In these assays, allele specific oligonucleotides with different markers, for example, fluorescent dyes, are used. The ligation products are then analyzed together, for example, by electrophoresis on an automatic DNA sequencer distinguishing markers by size and alleles by fluorescence. In the assay by Grossman et al., 1994, mobility is further modified by the presence of a non-nucleotide mobility modifier on one of the oligonucleotides.

A further modification of the ligation assay has been termed the dye-labeled oligonucleotide ligation (DOL) assay (U.S. Pat. No. 5,945,283; Chen et al., Genome Res., 8:549-556, 1998). DOL combines PCR and the oligonucleotide ligation reaction in a two-stage thermal cycling sequence with fluorescence resonance energy transfer (FRET) detection. In the assay, labeled ligation oligonucleotides are designed to have annealing temperatures lower than those of the amplification primers. After amplification, the temperature is lowered to a temperature where the ligation oligonucleotides can anneal and be ligated together. This assay requires the use of a thermostable ligase and a thermostable DNA polymerase without 5′ nuclease activity. Because FRET occurs only when the donor and acceptor dyes are in close proximity, ligation is inferred by the change in fluorescence.

In another method for the detection of SNPs termed minisequencing, the target-dependent addition by a polymerase of a specific nucleotide immediately downstream (3′) to a single primer is used to determine which allele is present (U.S. Pat. No. 5,846,710). Using this method, several SNPs can be analyzed in parallel by separating locus specific primers on the basis of size via electrophoresis and determining allele specific incorporation using labeled nucleotides.

Determination of individual SNPs using solid phase minisequencing has been described by Syvanen et al., Am. J. Hum. Genet., 52:46-59, 1993. In this method, the sequence including the polymorphic site is amplified by PCR using one amplification primer which is biotinylated on its 5′ end. The biotinylated PCR products are captured in streptavidin-coated microtitration wells, the wells washed, and the captured PCR products denatured. A sequencing primer is then added whose 3′ end binds immediately prior to the polymorphic site, and the primer is elongated by a DNA polymerase with one single labeled DNTP complementary to the nucleotide at the polymorphic site. After the elongation reaction, the sequencing primer is released and the presence of the labeled nucleotide detected. Alternatively, dye labeled dideoxynucleoside triphosphates (ddNTPs) can be used in the elongation reaction (U.S. Pat. No. 5,888,819; Shumaker et al., Human Mut., 7:346-354, 1996). In this method, incorporation of the ddNTP is determined using an automatic gel sequencer.

Minisequencing has also been adapted for use with microarrays (Shumaker et al., Human Mut., 7:346-354, 1996). In this case, elongation (extension) primers are attached to a solid support such as a glass slide. Methods for construction of oligonucleotide arrays are well known to those of ordinary skill in the art and can be found, for example, in Nature Genetics, Suppl., Vol. 21, January, 1999. PCR products are spotted on the array and allowed to anneal. The extension (elongation) reaction is carried out using a polymerase, a labeled DNTP and noncompeting ddNTPs. Incorporation of the labeled DNTP is then detected by the appropriate means. In a variation of this method suitable for use with multiplex PCR, extension is accomplished with the use of the appropriate labeled ddNTP and unlabeled ddNTPs (Pastinen et al., Genome Res., 7:606-614, 1997).

Solid phase minisequencing has also been used to detect multiple polymorphic nucleotides from different templates in an undivided sample (Pastinen et al., Clin. Chem., 42:1391-1397, 1996). In this method, biotinylated PCR products are captured on the avidin-coated manifold support and rendered single stranded by alkaline treatment. The manifold is then placed serially in four reaction mixtures containing extension primers of varying lengths, a DNA polymerase and a labeled ddNTP, and the extension reaction allowed to proceed. The manifolds are inserted into the slots of a gel containing formamide which releases the extended primers from the template. The extended primers are then identified by size and fluorescence on a sequencing instrument.

Fluorescence resonance energy transfer (FRET) has been used in combination with minisequencing to detect SNPs (U.S. Pat. No. 5,945,283; Chen et al., Proc. Natl. Acad. Sci. USA, 94:10756-10761, 1997). In this method, the extension primers are labeled with a fluorescent dye, for example fluorescein. The ddNTPs used in primer extension are labeled with an appropriate FRET dye. Incorporation of the ddNTPs is determined by changes in fluorescence intensities.

The above discussion of methods for the detection of SNPs is exemplary only and is not intended to be exhaustive. Those of ordinary skill in the art will be able to envision other methods for detection of SNPs that are within the scope and spirit of the present invention.

In one embodiment the present invention provides a method for diagnosing a genetic predisposition for a disease preferably, preferably hypertension, end stage renal disease due to hypertension, non-insulin dependent diabetes mellitus, end stage renal disease due to non-insulin dependent diabetes mellitus, breast cancer, lung cancer, or prostate cancer. In this method, a biological sample is obtained from a subject. The subject can be a human being or any vertebrate animal. The biological sample must contain nucleic acid (polynucleotides) and preferably genomic DNA. Samples that do not contain genomic DNA, for example, pure samples of mammalian red blood cells, are not preferred for use in the method. The form of the nucleic acid may vary such that the use of DNA, cDNA, RNA or mRNA is contemplated within the scope of the method. The polynucleotide is then analyzed to detect the presence or absence of a genetic variant where such variant is associated with a genetic predisposition to a disease, condition or disorder, preferably hypertension, end stage renal disease due to hypertension, non-insulin dependent diabetes mellitus, end stage renal disease due to non-insulin dependent diabetes mellitus, breast cancer, lung cancer, or prostate cancer. In one embodiment, the genetic variant is preferably located at position 2548, 2684, 2575, 1272, 2841, 2843 or 3556 of SEQ ID NO: 1. In another embodiment, the genetic variant is G2548→A, C2684→T, C2575→T, C1272 deletion, T2841→A, G2843→T or G3556→T or the complements thereof, i.e. C2548′→T C2684′→A, G2575′→A, G1272′ deletion, A2841′→T, or C2843′→A. As used herein, a “′” following a position number indicates the position on the template (−) strand that corresponds to the same position on the coding (+) strand. Thus 2548′ is the position on the template strand that corresponds to position 2548 on the coding strand. Any method capable of detecting a genetic variant, including any of the methods previously discussed, can be used. Suitable methods include, but are not limited to, those methods based on sequencing, mini sequencing, hybridization, restriction fragment analysis, oligonucleotide ligation, or allele specific PCR.

The present invention is also directed to an isolated nucleic acid sequence of at least 10 contiguous nucleotides from SEQ ID NO: 1 or the complement of SEQ ID NO 1 containing at least one single nucleotide polymorphism site associated with a disease, condition or disorder, preferably, hypertension, end stage renal disease due to hypertension, non-insulin dependent diabetes mellitus, end stage renal disease due to non-insulin dependent diabetes mellitus, breast cancer, lung cancer, or prostate cancer. In one embodiment, the polymorphic site is preferably at position 2548, 2684, 2575, 1272, 2841, 2843 or 3556 of SEQ ID NO: 1. In another embodiment, the polymorphic site contains a genetic variant, preferably, the genetic variants G2548→A, C2684→T, C2575→T, C1272 deletion, T2841→A, G2843→T or G3556→T or the complements thereof, i.e. C2548′→T C2684′→A, G2575′→A, G1272′ deletion, A2841′→T, or C2843′→A. In yet another embodiment, the polymorphic site, which may or may not also include a genetic variant, is located at the 3′ end of the polynucleotide. In still another embodiment, the polynucleotide further contains a detectable marker. Suitable markers include, but are not limited to, radioactive labels, such as radionuclides, fluorophores or fluorochromes, peptides, enzymes, antigens, antibodies, vitamins or steroids.

The present invention also includes kits for the detection of polymorphisms associated with diseases, conditions or disorders, preferably, preferably hypertension, end stage renal disease due to hypertension, non-insulin dependent diabetes mellitus, end stage renal disease due to non-insulin dependent diabetes mellitus, breast cancer, lung cancer, or prostate cancer. The kits contain, at a minimum, at least one polynucleotide of at least 10 contiguous nucleotides of SEQ ID NO 1 or the complement of SEQ ID NO: 1 containing at least one single nucleotide polymorphism site, preferably at position 2548, 2684, 2575, 1272, 2841, 2843 or 3556 of SEQ ID NO: 1. Alternatively the 3′ end of the polynucleotide is immediately 5′ to a polymorphic site, preferably located at position 2548, 2684, 2575, 1272, 2841, 2843 or 3556 of SEQ ID NO: 1. In one embodiment, the polymorphic site contains a genetic variant, preferably G2548′→A, C2684′→T, C2575′→T, C1272′ deletion, T2841→A, G2843′→T or G3556→T or the complements thereof, i.e. C2548′→T C2684′→A, G2575′→A, G1272′ deletion, A2841′→T, or C2843′→A. In still another embodiment, the genetic variant is located at the 3′ end of the polynucleotide. In yet another embodiment, the polynucleotide of the kit contains a detectable label. Suitable labels include, but are not limited to, radioactive labels, such as radionuclides, fluorophores or fluorochromes, peptides, enzymes, antigens, antibodies, vitamins or steroids.

In addition, the kit may also contain additional materials for detection of the polymorphisms. For example, and without limitation, the kits may contain buffer solutions, enzymes, nucleotide triphosphates, and other reagents and materials necessary for the detection of genetic polymorphisms. Additionally, the kits may contain instructions for conducting analyses of samples for the presence of polymorphisms and for interpreting the results obtained.

In yet another embodiment the present invention provides a method for designing a treatment regime for a patient having a disease, condition or disorder, preferably hypertension, end stage renal disease due to hypertension, non-insulin dependent diabetes mellitus, end stage renal disease due to non-insulin dependent diabetes mellitus, breast cancer, lung cancer, or prostate cancer, caused either directly or indirectly by the presence of one or more single nucleotide polymorphisms preferably G2548→A, C2684→T, C2575→T, C1272 deletion, T2841→A, G2843→T or G3556→T or the complements thereof, i.e. C2548′→T C2684′→A, G2575′→A, G1272′ deletion, A2841′→T, or C2843′→A. In this method genetic material from a patient, for example, DNA, cDNA, RNA or mRNA is screened for the presence of one or more SNPs associated with the disease of interest. Depending on the type and location of the SNP, a treatment regime is designed to counteract the effect of the SNP. For example and without limitation, genetic material from a patient suffering from end-stage renal disease (ESRD) can be screened for the presence of SNPs associated with ESRD. If one or more of the SNPs found disrupt a sequence in the ecNOS promoter region, such that there is less nitric oxide (NO) produced in tissues such as endothelial cells, a treatment, such as oral administration of L-arginine, a substrate for nitric oxide production, is devised to counteract the decreased nitric oxide production due to the SNP.

Alternatively, information gained from analyzing genetic material for the presence of polymorphisms can be used to design treatment regimes involving gene therapy. For example, detection of a polymorphism that either affects the expression of a gene or results in the production of a mutant protein can be used to design an artificial gene to aid in the production of normal, wild type protein or help restore normal gene expression. Methods for the construction of polynucleotide sequences encoding proteins and their associated regulatory elements are well know to those of ordinary skill in the art ((Ausubel et al., Short Protocols in Molecular Biology, 3^{rd} ed, John Wiley & Sons, 1995; Sambrook et al., Molecular Cloning, Cold Spring Harbor Laboratory Press, 1989; and Davis et al., Basic Methods in Molecular Biology, Elsevier Science Publishing, 1986)). Once designed, the gene can be placed in the individual by any suitable means known in the art (Gene Therapy Technologies, Applications and Regulations, Meager, ed., Wiley, 1999; Gene Therapy: Principles and Applications, Blankenstein, ed., Birkhauser Verlag, 1999; Jain, Textbook of Gene Therapy, Hogrefe and Huber, 1998).

The present invention is also useful in designing prophylactic treatment regimes for patients determined to have a genetic predisposition to a disease, condition or disorder, preferably, preferably hypertension, end stage renal disease due to hypertension, non-insulin dependent diabetes mellitus, end stage renal disease due to non-insulin dependent diabetes mellitus, breast cancer, lung cancer, or prostate cancer, due to the presence of one or more single nucleotide polymorphisms preferably G2548→A, C2684→T, C2575→T, C1272 deletion, T2841→A, G2843→T or G3556→T or the complements thereof, i.e. C2548→T C2684→A, G2575→A, G1272′ deletion, A2841′→T, or C2843′→A. In this embodiment, genetic material, such as DNA, cDNA, RNA or mRNA, is obtained from a patient and screened for the presence of one or more SNPs associated either directly or indirectly to a disease, condition, disorder or other pathological condition. Based on this information, a treatment regime can be designed to decrease the risk of the patient developing the disease. Such treatment can include, but is not limited to, surgery, the administration of pharmaceutical compounds or nutritional supplements, and behavioral changes such as improved diet, increased exercise, reduced alcohol intake, smoking cessation, etc.

For example, and without limitation, a patient with an increased risk of developing renal disease due to the presence of a SNP in the ecNOS promoter could be given treatment to increase the production of nitric oxide (NO) by, for example the oral administration of L-arginine, thus reducing the risk of developing renal disease.

### EXAMPLES

### Example 1

### G to A Transition at Position 2548

Amplification of eNOS Promoter Genomic DNA

Leukocytes were obtained from human whole blood collected with EDTA. Genomic DNA was purified from the collected leukocytes using standard protocols well known to those of ordinary skill in the art of molecular biology (Ausubel et al., Short Protocols in Molecular Biology, 3^{rd} ed, John Wiley & Sons, 1995; Sambrook et al., Molecular Cloning, Cold Spring Harbor Laboratory Press, 1989; and Davis et al., Basic Methods in Molecular Biology, Elsevier Science Publishing, 1986).

DNA comprising the eNOS promoter region was amplified by the polymerase chain reaction (PCR). Twenty-five ng of leukocyte genomic DNA was used as template for each PCR amplification. Twenty-five microliters of an aqueous solution of genomic DNA (1 ng/ul) was dispensed to the wells of a 96-well plate, and dried down at 70° C. for 15 minutes. The DNA was rehydrated with 7 ul of ultra-pure but not autoclaved water (Milli-Q, Millipore Corp., Bedford Mass.). PCR conditions were as follows: 5 minutes at 94° C., followed by 45 cycles, where each cycle consisted of 94° C. for 45 seconds to denature the double-stranded DNA, then 64° C. for 45 seconds for specific annealing of primers to the single-stranded DNA, then 72° C. for 45 seconds for extension. After the 45th cycle, the reaction mixture was held at 72° C. for 10 minutes for a final extension reaction. The PCR reaction contained a total volume of 20 microliters (ul), and consisted of 10 ul of a pre-made PCR reaction mix (Sigma “JumpStart Ready Mix with RED Taq Polymerase” Sigma Chemical, St. Louis, Mo.). Primers at 10 μM were diluted to a final concentration of 0.3 μM in the PCR reaction mix. The forward primer was 5′ gagtctggccaacacaaatcc 3′ (SEQ ID NO: 3) and the reverse primer was 5′ ctctagggtcatgcaggttct c 3′ (SEQ ID NO: 4). The primers amplified the region spanning nucleotides 2356 to 2559, inclusive of SEQ ID NO: 1. Post-PCR clean-up was performed prior to submission of PCR product to pyrosequencing.

Sequencing of PCR Product

Pyrosequencing is a method of sequencing DNA by synthesis, where the addition of one of the four dNTPs that correctly matches the complementary base on the template strand is detected. Detection occurs via utilization of the pyrophosphate molecules liberated upon base addition to the elongating synthetic strand. The pyrophosphate molecules are used to make ATP, which in turn drives the emission of photons in a luciferin/luciferase reaction, and these photons are detected by the pyrosequencer.

A Luc96 Pyrosequencer (Pyrosequencing AB, Uppsala Sweden) was used under default operating conditions supplied by the manufacturer. Sequencing primers were designed to anneal within 5 bases of the polymorphism. Patient genomic DNA was subject to PCR using amplifying primers that amplify an approximately 200 base pair amplicon containing the polymorphisms of interest as described in Example 1. One of the amplifying primers, whose orientation is opposite to that of the sequencing primer, was biotinylated. This allowed selection of single stranded template for pyrosequencing, whose orientation was complementary to the sequencing primer. Amplicons prepared from genomic DNA were isolated by binding to streptavidin-coated magnetic beads. After denaturation in NaOH, the biotinylated strands were separated from their complementary strands using magnetic beads (DYNAL, Olso, Norway). After washing the magnetic beads, the biotinylated template strands still bound to the beads were transferred into 96-well plates. The sequencing primers were added, annealing was carried out at 95° C. for 2 minutes, and plates placed in the pyrosequencer.

The enzymes, substrates and dNTPs used for synthesis and pyrophosphate detection were added to the instrument immediately prior to sequencing. The Luc96 software requires definition of a program of adding the four dNTPs that is specific for the location of the sequencing primer, the DNA composition flanking the SNP, and the two possible alleles at the polymorphic locus. The order of adding bases generates theoretical outcomes of light intensity patterns for each of the two possible homozygous states and the single heterozygous state. The Luc96 software then compares the actual outcome to the theoretical outcome and calls a genotype for each well. Each sample is also assigned one of three confidence scores: pass, uncertain, or fail. The results for each plate were output as a text file and processed in Excel using a Visual Basic program to generate a report of genotype and allele frequencies for the various disease and population cell groupings represented on the 96 well plate.

Bioinformatics

Prediction of potential transcription binding factor sites was performed using a commercially available software program [GENOMATIX MatInspector Professional release 4.2, February, 2000; http://genomatix.gsf.de/cqi-bin/matinspector/matinspector.pl;. Quandt K et al., Nucleic Acids Res 23: 4878-4884 (1995)].

DNA Samples

Cases consisted of patients with essential hypertension or non-insulin dependent diabetes mellitus (NIDDM) (type II diabetes mellitus), but without evidence of renal disease (<2+ proteinuria on random urinalysis; serum creatinine less than or equal to 1.5 mg/dl). Samples were obtained from indigent-care St. Louis-area hospitals between 1994 and 1996.

Patients with end-stage renal disease (ESRD) due to hypertension (ESRD/HTN) or due to NIDDM (ESRD/NIDDM) were hemodialysis patients with either hypertension only, or NIDDM (with or without hypertension), being treated in approximately 40 dialysis units in the southeastern US. Their samples were obtained in 1995.

Disease-free controls were healthy plasma donors from cities in the central and eastern United States, with normal serum creatinine (less than or equal to 1.5 mg/dl). Controls were screened routinely to ensure the absence of any infectious diseases. Control plasma donors could not be taking insulin or other medication, except for a single anti-hypertensive at a low dose. Thus, controls could have mild essential hypertension, but no renal disease, and no NIDDM.

Cases and controls were matched for ethnicity, gender, and sex, but not age.

Statistics

Allele and genotype frequencies were stratified on the combination of race and gender (hereinafter referred to as a ‘cell’) and then matched to controls for an association study. Three statistics, a point estimate, 95% confidence interval, and a likelihood (p-value), were calculated for each combination of cell and disease. A simple odds ratio was used as the point estimate of association. In the case where a cell count was 0, the Haldane correction was used. This consists of adding 0.5 to each cell prior to calculations. The 95% confidence intervals were calculated using the asymptotic method. P-values for differences in allele or genotype frequencies were calculated using Fisher's exact test, using a two-sided alternative to the null hypothesis. All calculations were done using the SAS suite of statistical software, version 8.1 (SAS Institute, Cary, N.C.)

Results

Using the methods described above, a substitution mutation (transition) was found in which the G found in the reference sequence (SEQ ID NO: 1) was replaced with an A. Data analysis produced the following results.

TABLE 1
ALLELE FREQUENCES

| | | CHROMOSOMES | G | % | A | % |
|---|---|---|---|---|---|---|
| Disease | Cell | | | | | |
| Controls | Black men | 1340 | 280 | 21% | 1060 | 79% |
| | Black women | 1380 | 159 | 12% | 1221 | 88% |
| | White men | 1412 | 402 | 28% | 1010 | 72% |
| | White women | 1482 | 532 | 36% | 950 | 64% |
| Hypertension | Black men | 568 | 139 | 24% | 429 | 76% |
| | Black women | 348 | 86 | 25% | 262 | 75% |
| | White men | 562 | 150 | 27% | 412 | 73% |
| | White women | 130 | 46 | 35% | 84 | 65% |
| ESRD due to HTN | Black men | 568 | 261 | 46% | 307 | 54% |
| | Black women | 440 | 196 | 45% | 244 | 55% |
| | White men | 306 | 108 | 35% | 198 | 65% |
| | White women | 284 | 126 | 44% | 158 | 56% |
| NIDDM | Black men | 530 | 161 | 30% | 369 | 70% |
| | Black women | 368 | 135 | 37% | 233 | 63% |
| | White men | 472 | 136 | 29% | 336 | 71% |
| | White women | 86 | 26 | 30% | 60 | 70% |
| ESRD due to NIDDM | Black men | 512 | 48 | 9.4% | 464 | 91% |
| | Black women | 496 | 78 | 16% | 418 | 84% |
| | White men | 426 | 174 | 41% | 252 | 59% |
| | White women | 392 | 115 | 29% | 277 | 71% |

TABLE 2
GENOTYPE FREQUENCIES

| | | Total ‘n’ | G/G | % | G/A | % | A/A | % |
|---|---|---|---|---|---|---|---|---|
| Disease | Cell | | | | | | | |
| Controls | Black men | 670 | 35 | 5.2% | 210 | 31.3% | 425 | 63.4% |
| | Black women | 690 | 6 | 0.9% | 147 | 21.3% | 537 | 77.8% |
| | White men | 706 | 62 | 8.8% | 278 | 39.4% | 366 | 51.8% |
| | White women | 741 | 103 | 13.9% | 326 | 44.0% | 312 | 42.1% |
| Hypertension | Black men | 284 | 3 | 1.1% | 133 | 46.8% | 148 | 52.1% |
| | Black women | 174 | 0 | 0.0% | 86 | 49.4% | 88 | 50.6% |
| | White men | 281 | 12 | 4.3% | 126 | 44.8% | 143 | 50.9% |
| | White women | 65 | 5 | 7.7% | 36 | 55.4% | 24 | 36.9% |
| ESRD due to HTN | Black men | 284 | 0 | 0.0% | 261 | 91.9% | 23 | 8.1% |
| | Black women | 220 | 3 | 1.4% | 190 | 86.4% | 27 | 12.3% |
| | White men | 153 | 0 | 0.0% | 108 | 70.6% | 45 | 29.4% |
| | White women | 142 | 6 | 4.2% | 114 | 80.3% | 22 | 15.5% |
| NIDDM | Black men | 265 | 2 | 0.8% | 157 | 59.2% | 106 | 40.0% |
| | Black women | 184 | 0 | 0.0% | 135 | 73.4% | 49 | 26.6% |
| | White men | 236 | 7 | 3.0% | 122 | 51.7% | 107 | 45.3% |
| | White women | 43 | 3 | 7.0% | 20 | 46.5% | 20 | 46.5% |
| ESRD due to | Black men | 256 | 6 | 2.3% | 36 | 14.1% | 214 | 83.6% |
| NIDDM | Black women | 248 | 8 | 3.2% | 62 | 25.0% | 178 | 71.8% |
| | White men | 213 | 27 | 12.7% | 120 | 56.3% | 66 | 31.0% |
| | White women | 196 | 16 | 8.2% | 83 | 42.3% | 97 | 49.5% |

The susceptibility allele, the odds ratio (OR), 95% confidence interval, and p-value are given in Table 3. An odds ratio of 1.5 was chosen a priori as the threshold of practical significance based on the recommendation of Austin H et al. (Epidemiol. Rev. 16:65-76, 1994). “ . . . [E]pidemiology in general and case-control studies in particular are not well suited for detecting weak associations (odds ratios <1.5)[p. 66].”

This threshold of 1.5 is supported by our data, considering p<0.05 as the level of significance. All odds ratios attaining p<0.05 or better are underlined below. (Scientific notation is used in some entries below, e.g. 2.9E−9=2.9×10⁻⁹).

An example of an odds ratio calculation is given below:

Hypertension: Black women

| | Cases | Controls |
|---|---|---|
| G | 86 | 159 |
| A | 262 | 1221 |

In this example, the odds ratio that the G allele is the susceptibility allele for black women with hypertension is (86)(1221)/(262)(159)=2.5.

TABLE 3
ALLELE-SPECIFIC ODDS RATIOS

| | | Risk | Odds | | P |
|---|---|---|---|---|---|
| | | Allele | Ratio | 95% CI | Value |
| Disease | Cell | | | | |
| Hypertension | Black men | G | 1.2 | 1.0-1.5 | 0.09 |
| | Black women | G | 2.5 | 1.9-3.4 | 2.9E−9 |
| | White men | A | 1.1 | 0.9-1.4 | 0.44 |
| | White | A | 1.0 | 0.7-1.5 | 1.0 |
| | women | | | | |
| ESRD due to | Black men | G | 2.6 | 2.0-3.4 | 4.0E−14 |
| HTN^{†} | Black women | G | 2.4 | 1.8-3.3 | 7.3E−9 |
| | White men | G | 1.5 | 1.1-2.0 | 0.01 |
| | White | G | 1.5 | 0.9-2.2 | 0.09 |
| | women | | | | |
| NIDDM | Black men | G | 1.7 | 1.3-2.1 | 0.00002 |
| | Black women | G | 4.4 | 3.4-5.8 | 1.5E−26 |
| | White men | G | 1.0 | 0.8-1.3 | 0.90 |
| | White | A | 1.3 | 0.8-2.1 | 0.30 |
| | women | | | | |
| ESRD due to | Black men | A | 4.2 | 3.0-6.0 | 7.9E−18 |
| NIDDM^{‡} | Black women | A | 3.1 | 2.3-4.3 | 2.2E−12 |
| | White men | G | 1.7 | 1.3-2.3 | 0.00019 |
| | White | A | 1.0 | 0.6-1.7 | 0.89 |
| | women | | | | |

^{†}Odds ratios calculated using patients with hypertension as controls
^{‡}Odds ratios calculated using patients with NIDDM as controls

The genotype-specific odds ratios are given in Table 4. In Table 4, the susceptibility allele (S) is indicated. The alternative allele at this locus is defined as the protective allele (P). Also presented is the odds ratio (OR) for the SS and SP genotypes. The odds ratio for the PP genotype is 1, since it is the reference group, and is not presented separately. The 95% confidence interval (C.I.) is also given, in parentheses. An odds ratio of 1.5 was chosen as the threshold of significance based on the recommendation of Austin H et al. (Epidemiol. Rev. 16:65-76, 1994). “. . . [E]pidemiology in general and case-control studies in particular are not well suited for detecting weak associations (odds ratios <1.5)[p. 66].”

Odds ratios attaining 1.5 are high-lighted below. Where Haldane's zero cell correction was used, the odds ratio is so indicated with a superscript “H”.

An example is worked below, assuming that G is the susceptibility allele (S), and A is the protective allele (P).

Black women: ESRD due to HTN

| | Cases | Controls |
|---|---|---|
| GG (SS) | 3 | 0 |
| GA (SP) | 190 | 86 |
| AA (PP) | 27 | 88 |

Applying Haldane's correction only where the denominator of the odds ratio contains a 0, the SS odds ratio is (3.5)(88.5)/(27.5)(0.5)=22.5 while the SP odds ratio is (190)(88)/(27)(86)=7.2

TABLE 4
GENOTYPE-SPECIFIC ODDS RATIOS

| | | RISK | SS | | SP | |
|---|---|---|---|---|---|---|
| | | ALLELE | O.R. | 95% C.I. | O.R. | 95% C.I. |
| Disease | Cell | | | | | |
| Hypertension | Black men | G | 0.2 | 0.1-0.8 | 1.8 | 1.4-2.4 |
| | Black women | G | 0.0 | | 3.6 | 2.5-5.1 |
| | White men | A | 2.0 | 1.1-3.9 | 2.3 | 1.8-3.1 |
| | White women | A | 1.6 | 0.6-4.3 | 2.3 | 1.3-3.9 |
| ESRD due to HTN^{†} | Black men | G | 0.0 | | 12.6 | 7.8-20.5 |
| | Black women | G | 22.5^{H} | 0.4-1325.4 | 7.2 | 4.4-11.9 |
| | White men | G | 0.0 | | 2.7 | 1.8-4.2 |
| | White women | G | 1.3 | 0.3-4.9 | 3.5 | 1.7-6.9 |
| NIDDM | Black men | G | 0.2 | 0.1-0.8 | 3.0 | 2.2-4.0 |
| | Black women | G | 0.0 | | 10.1 | 6.9-14.6 |
| | White men | G | 0.4 | 0.2-0.9 | 1.5 | 1.1-2.0 |
| | White women | A | 2.2 | 0.6-7.6 | 2.1 | 1.1-4.0 |
| ESRD due to NIDDM^{‡} | Black men | A | 0.7 | 0.1-3.4 | 0.1 | 0.0-0.1 |
| | Black women | A | 0.0 | | 0.0 | |
| | White men | G | 6.3 | 2.6-15.2 | 1.6 | 1.1-2.4 |
| | White women | A | 0.9 | 0.2-3.4 | 0.8 | 0.4-1.5 |

^{†}Odds ratios calculated using patients with hypertension as controls
^{‡}Odds ratios calculated using patients with NIDDM as controls

Hardy-Weinberg analysis was conducted on the control samples. Hardy-Weinberg equilibrium is a term used to describe the distribution of genotypes at a bialleleic locus in a stable population without recent genetic admixture, drift, or selection pressure. The equilibrium distribution is the binomial expansion of the two allele frequencies, p and q=1−p, i.e. (p+q)²=p²+2pq+q²=1.

The control samples were in good agreement with Hardy-Weinberg equilibrium, as follows:

A frequency of 0.12 for the G allele (“p”) and 0.88 for the A allele (“q”) among black female control individuals predicts genotype frequencies of 1.4% GIG, 21.2% G/A, and 77.4% A/A at Hardy-Weinberg equilibrium (p²+2pq+q²=1). The observed genotype frequencies were 0.9% G/G, 21.3% G/A, and 77.8% A/A, in excellent agreement with those predicted for Hardy-Weinberg equilibrium. The chi-square statistic for a test of disequilibrium was 1.1, which has a p-value of 0.58, with 2 degrees of freedom. Thus, the observed genotype frequencies do not deviate significantly from Hardy-Weinberg equilibrium (HWE).

A frequency of 0.21 for the G allele (“p”) and 0.79 for the A allele (“q”) among black male control individuals predicts genotype frequencies of 4.4% G/G, 33.2% G/A, and 62.4% A/A at Hardy-Weinberg equilibrium (p²+2pq+q²=1). The observed genotype frequencies were 5.2% G/G, 31.3% G/A, and 63.5% A/A, in excellent agreement with those predicted for Hardy-Weinberg equilibrium. The chi-square statistic for a test of disequilibrium was 1.3, which has a p-value of 0.51 with 2 degrees of freedom. Thus, the observed genotype frequencies do not deviate significantly from Hardy-Weinberg equilibrium.

A frequency of 0.36 for the G allele (“p”) and 0.64 for the A allele (“q”) among white female control individuals predicts genotype frequencies of 13.0% G/G, 46.1% G/A, and 40.9% A/A at Hardy-Weinberg equilibrium (p²+2pq+q²=1). The observed genotype frequencies were 13.9% GIG, 44.0% G/A, and 42.1% A/A, in good agreement with those predicted for Hardy-Weinberg equilibrium. The chi-square statistic for a test of disequilibrium was 0.96, which has a p-value of 0.60 with 2 degrees of freedom. Thus, the observed genotype frequencies do not deviate significantly from Hardy-Weinberg equilibrium.

A frequency of 0.28 for the G allele (“p”) and 0.72 for the A allele (“q”) among white male control individuals predicts genotype frequencies of 7.8% G/G, 40.3% G/A, and 51.9% A/A at Hardy-Weinberg equilibrium (p²+2pq+q²=1). The observed genotype frequencies were 8.8% G/G, 39.4% G/A, and 51.8% A/A, in excellent agreement with those predicted for Hardy-Weinberg equilibrium. The chi-square statistic for a test of disequilibrium was 0.7, which has a p-value of 0.7 with 2 degrees of freedom. Thus, the observed genotype frequencies do not deviate significantly from Hardy-Weinberg equilibrium.

Hypertension and NIDDM are necessary but not sufficient to develop ESRD. Patients with hypertension are at approximately a 5% lifetime risk of ESRD, while patients with NIDDM are at about a 20% lifetime risk. Therefore hypertension and NIDDM can be considered as intermediate phenotypes; clinically diseased compared to the average population, yet healthier than hypertensive or diabetic patients with ESRD.

In order to detect a dosage effect of the G2548→A polymorphism, a progressive disease model for calculating odds ratios was used. The odds ratio for patients with hypertension alone or NIDDM alone relative to normal controls represents a baseline measurement for each underlying disease. Next, calculating odds ratios for ESRD patients by comparing them to individuals with just the primary disease but no kidney disease (ie HTN or NIDDM) can be useful in dissecting which alleles are necessary for progression to end-stage kidney failure.

Using an allele-specific odds ratio of 1.5 or greater as a practical level of significance (see Austin H. et al., discussed above), the following observations, which are summarized in Table 5, can be made.

For black women with hypertension, the odds ratio for the G allele was 2.5 [(95% CI, 1.9-3.4), p<2.9E−9]. The odds ratio for the homozygote (GIG) was less than 1.0, while the odds ratio for the heterozygote (G/A) was 3.6 (95% CI, 2.5-5.1). These data suggest that the G allele acts in a co-dominant manner in this patient population. These data further suggest that the ecNOS gene is significantly associated with hypertension alone in black women, i.e. abnormal activity of the ecNOS gene predisposes black women to hypertension.

For black women with ESRD due to hypertension, the odds ratio for the G allele was 2.4 [(95% CI, 1.8-3.3), p<7.3E−9], compared to black women with hypertension alone. The odds ratio for the homozygote (GIG) was 22.5^{H} [the superscript “H” indicates the Haldane correction was employed] (95% CI, 0.4-1325.4). The odds ratio for the heterozygote (G/A) was 7.2 (95% CI, 4.4-11.9). These data suggest that the G allele acts in a dominant manner in this patient population with a greater than additive effect of allele dosage [22.5>13.4=(7.2+7.2−1.0)] (Goldstein A M and Andrieu N, Monogr. Natl. Cancer Inst. 26: 49-54, 1999). These data further suggest that the ecNOS gene is significantly associated with ESRD due to hypertension in black women, i.e. abnormal activity of the ecNOS gene predisposes black women with hypertension to ESRD.

For black men with ESRD due to hypertension, the odds ratio for the G allele was 2.6 [(95% CI, 2-3.4), p<4.0E-14], compared to black men with hypertension alone. The odds ratio for the homozygote (G/G) was less than 1.0, while the odds ratio for the heterozygote (G/A) was 12.6 (95% CI, 7.8-20.5). These data suggest that the G allele acts in a co-dominant manner in this patient population. These data further suggest that the ecNOS gene is significantly associated with ESRD due to hypertension in black men, i.e. abnormal activity of the ecNOS gene predisposes black men with hypertension to ESRD.

For white men with ESRD due to hypertension, the odds ratio for the G allele was 1.5 [(95% CI, 1.1-2.0), p=0.01], compared to white men with hypertension alone. The odds ratio for the homozygote (G/G) was less than 1.0, while the odds ratio for the heterozygote (G/A) was 2.7 (95% CI, 1.8-4.2). These data suggest that the G allele acts in a co-dominant manner in this patient population. These data further suggest that the ecNOS gene is significantly associated with ESRD due to hypertension in white men, i.e. abnormal activity of the ecNOS gene predisposes white men with hypertension to ESRD.

For black men with NIDDM alone, the odds ratio for the G allele was 1.7 [(95% CI, 1.3-2.1), p<0.00002]. The odds ratio for the homozygote (GIG) was less than 1.0, while the odds ratio for the heterozygote (G/A) was 3.0 (95%CI, 2.2-4). These data suggest that the G allele acts in a co-dominant manner in this patient population. These data further suggest that the ecNOS gene is significantly associated with NIDDM in black men, i.e. abnormal activity of the ecNOS gene predisposes black men to NIDDM.

For black men with ESRD due to NIDDM, the odds ratio for the A allele was 4.2 [(95% CI, 3.0-6.0), p<7.9E-18], compared to black men with NIDDM alone. Data were not sufficient to generate genotypic odds ratios of 1.5 or greater. These data further suggest that the ecNOS gene is significantly associated with ESRD due to NIDDM in black men, i.e. abnormal activity of the ecNOS gene predisposes black men with NIDDM to ESRD.

For black women with NIDDM, the odds ratio for the G allele was 4.4 [(95% CI, 3.4-5.8), p<1.5E−26]. The odds ratio for the homozygote (G/G) was less than 1.0, while the odds ratio for the heterozygote (G/A) was 10.1 (95% CI, 6.9-14.6). These data suggest that the G allele acts in a co-dominant manner in this patient population. These data further suggest that the ecNOS gene is significantly associated with NIDDM in black women, i.e. abnormal activity of the ecNOS gene predisposes black women to NIDDM.

For black women with ESRD due to NIDDM, the odds ratio for the A allele was 3.1 [(95% CI, 2.3-4.3), p<2.2E-12], compared to black women with NIDDM alone. Data were not sufficient to generate genotypic odds ratios of 1.5 or greater. These data further suggest that the ecNOS gene is significantly associated with ESRD due to NIDDM in black women, i.e. abnormal activity of the ecNOS gene predisposes black women with NIDDM to ESRD.

For white men with ESRD due to NIDDM the odds ratio for the G allele was 1.7 [(95% CI, 1.3-2.3), p<0.0002], compared to white men with NIDDM alone. The odds ratio for the homozygote (G/G) was 6.3 (95% CI, 2.6-15.2), while the odds ratio for the heterozygote (G/A) was 1.6 (95% CI, 1.1-2.4). These data suggest that the G allele acts in a dominant manner in this patient population, with a greater than multiplicative effect of allele dosage [6.3>2.56=(1.6)(1.6)]. These data further suggest that the ecNOS gene is significantly associated with ESRD due to NIDDM in white men, i.e. abnormal activity of the ecNOS gene predisposes white men with NIDDM to ESRD.

TABLE 5

SUSCEPTIBILITY ALLELE

| | CAUCASIAN | | AFRICAN-AMERICAN | |
|---|---|---|---|---|
| DISEASE | Men | Women | Men | Women |

| | | | | |
|---|---|---|---|---|
| HTN | A | A | G | G** |
| ESRD/HTN | G* | G | G** | G** |
| NIDDM | G | A | G* | G** |
| ESRD/NIDDM | G* | A | A** | A** |

**p < 5E−8;
*p < 0.05

According to commercially available software (GENOMATIX MatInspector Professional), the G2548→A SNP is predicted to have the following effects on transcription of the ecNOS gene.

One predicted effect is disruption of an NF-1 (nuclear factor 1) site (5′-AGATGGCACAGAACTACA-3′; SEQ ID NO: 5) beginning at position +2543 on the (+) strand. This polymorphism would result in replacement of the indicated G by an A. NF-1 sites occur relatively frequently in the genome: 4.11 occasions per 1000 base pairs of random genomic sequence in vertebrates. Since NF-1 is a positive transcriptional regulator disruption of its binding site is expected to result in a decreased rate of transcription of the ecNOS gene. If the rate of translation is tied to the level of messenger RNA, as is the case for most proteins, then less gene product (ecNOS enzyme) will be the result, ultimately leading to less nitric oxide (NO) produced in tissues such as endothelial cells in patients with the A allele.

The polymorphism also can cause disruption of an MYOD (myoblast determining factor) binding site, which consists of 5′-GCCATCTC-AG-3′ (SEQ ID NO: 6), ending at position +2540 on the (−) strand. Thus, this polymorphism results in replacement of the indicated C by a T on the (−) strand, since T is complementary to the polymorphic base, A, at this position on the (+) strand. MYOD binding sites are less frequent than NF1 sites, occurring 0.96 times per 1000 base pairs of random genomic sequence. MYOD is increasingly recognized as a potent transcriptional activator of more tissues than merely those destined to become skeletal muscle, in which context it was originally discovered. This association suggests an unexpected biochemical mechanism for diabetic or hypertensive renal failure, e.g. in black women, who express a higher frequency of the A allele. MYOD may operate in endothelial cells. It is possible that ecNOS production by smooth muscle cells, which are known to express MYOD, is important in regulation of renal blood flow and apoptosis of down-stream cellular elements.

Another predicted effect is disruption of an LMO2COM (complex of Lmo2 bound to Tal-1, E2A protein) binding site, which consists of the sequence 5′-CCTCAGATGGCA-3′ (SEQ ID NO: 7), beginning at position +2539 on the (+) strand. This polymorphism results in the replacement of the indicated G with an A. LMO2COM binding sites occur with a frequency of 1.11 times per 1000 base pairs of random genomic sequence, which is relatively frequent. The E2A protein is an adenoviral “early” protein, for which no cellular homolog is yet known.

Also predicted is the disruption of a TAL1ALPHAE47 (Tal-1alpha/E47 heterodimer) binding site, which consists of the sequence 5′-CCCCTCAGATGGCACA-3′ (SEQ ID NO: 8), beginning at position +2537 on the (+) strand. This polymorphism results in the replacement of the indicated G with an A. TAL1ALPHAE47 binding sites occur quite infrequently, at the rate of 0.14 times per 1000 base pairs of random genomic sequence in vertebrates. The less frequently that the binding site occurs in random genomic DNA, the more likely that the binding site is specifically involved in transcription of this gene. Association of disease with this site thus suggests a novel mechanism for ecNOS regulation in cells whose identity is not yet known, but which could include endothelial, smooth muscle, mesangial, or tubular epithelial cells, for example. The Tal-1beta (or alpha)/E47 heterodimer can behave as a transcriptional activator, so replacement of the indicated G with an A is predicted to result in a lower rate of transcription of the ecNOS gene and thus a lower level of nitric oxide production in tissues.

Another predicted effect is the disruption of a TAL1BETAE47 (Tal-1beta/E47 heterodimer) binding site, which consists of the sequence 5′-CCCCTCAGATGGCACA-3′ (SEQ ID NO: 8), beginning at position +2537 on the (+) strand. This polymorphism results in the replacement of the indicated G with an A. TAL1BETAE47 binding sites also occur quite rarely, at the rate of 0.11 times per 1000 base pairs of random genomic sequence. Association of disease with this site thus suggests a novel mechanism for ecNOS regulation in cells whose identity is not yet known, but which could include, for example, endothelial, smooth muscle, mesangial, or tubular epithelial cells. If Tal-1beta (or alpha)/E47 heterodimer behaves as a transcriptional activator, then replacement of the indicated G with an A is predicted to result in a lower rate of transcription of the ecNOS gene and thus a lower level of nitric oxide production in tissues.

### Example 2

### C to T Transition at Position 2684

Methods of DNA amplification, sequencing and data analysis were essentially as described in Example 1. A substitution mutation (transition) was found in which the C found at position 2684 in the reference sequence (SEQ ID NO: 1) was replaced with a T. Data analysis produced the following results.

TABLE 6
ALLELE FREQUENCIES

| | C | | T | |
|---|---|---|---|---|
| CONTROL | | | | |
| Black men (n = 84 chromosomes) | 10 | (12%) | 74 | (88%) |
| Black women (n = 74 chromosomes) | 18 | (24%) | 56 | (76%) |
| White men (n = 76 chromosomes) | 29 | (38%) | 47 | (62%) |
| White women (n = 94 chromosomes) | 29 | (31%) | 65 | (69%) |
| DISEASE | | | | |
| BREAST CANCER | | | | |
| Black women (n = 40 chromosomes) | 7 | (18%) | 33 | (82%) |
| White women (n = 38 chromosomes) | 12 | (32%) | 26 | (68%) |
| LUNG CANCER | | | | |
| Black men (n = 40 chromosomes) | 21 | (53%) | 19 | (48%) |
| Black women (n = 32 chromosomes) | 6 | (19%) | 26 | (81%) |
| White men (n = 40 chromosomes) | 17 | (43%) | 23 | (58%) |
| White women (n = 22 chromosomes) | 8 | (36%) | 14 | (64%) |
| PROSTATE CANCER | | | | |
| Black men (n = 40 chromosomes) | 9 | (23%) | 31 | (77%) |
| White men (n = 38 chromosomes) | 17 | (45%) | 21 | (55%) |
| NIDDM | | | | |
| Black men (n = 4 chromosomes) | 1 | (25%) | 3 | (75%) |
| Black women (n = 6 chromosomes) | 3 | (50%) | 3 | (50%) |
| White men (n = 8 chromosomes) | 0 | (0%) | 8 | (100%) |
| White women (n = 18 chromosomes) | 14 | (78%) | 4 | (22%) |
| ESRD due to NIDDM | | | | |
| Black men (n = 12 chromosomes) | 1 | (8%) | 11 | (92%) |
| Black women (n = 16 chromosomes) | 2 | (13%) | 14 | (88%) |
| White men (n = 10 chromosomes) | 2 | (20%) | 8 | (80%) |
| White women (n = 8 chromosomes) | 2 | (25%) | 6 | (75%) |
| HYPERTENSION (HTN) | | | | |
| Black men (n = 24 chromosomes) | 3 | (13%) | 21 | (88%) |
| Black women (n = 24 chromosomes) | 2 | (8%) | 22 | (92%) |
| White men (n = 22 chromosomes) | 7 | (32%) | 15 | (68%) |
| White women (n = 20 chromosomes) | 8 | (40%) | 12 | (60%) |
| ESRD due to HTN | | | | |
| Black men (n = 20 chromosomes) | 4 | (20%) | 16 | (80%) |
| Black women (n = 18 chromosomes) | 0 | (0%) | 18 | (100%) |
| White men (n = 18 chromosomes) | 5 | (28%) | 13 | (72%) |
| White women (n = 18 chromosomes) | 3 | (17%) | 15 | (83%) |
| MYOCARDIAL INFARCTION | | | | |
| White women (n = 14 chromosomes) | 5 | (36%) | 9 | (64%) |

TABLE 7
GENOTYPE FREQUENCIES

| | C/C | C/T | T/T |
|---|---|---|---|
| CONTROLS | | | |
| Black men (n = 42) | 0 (0%) | 10 (24%) | 32 (76%) |
| Black women (n = 37) | 2 (5%) | 14 (38%) | 21 (57%) |
| White men (n = 38) | 5 (13%) | 19 (50%) | 14 (37%) |
| White women (n = 47) | 2 (4%) | 25 (53%) | 20 (43%) |
| DISEASE | | | |
| BREAST CANCER | | | |
| Black women (n = 20) | 0 (0%) | 7 (35%) | 13 (65%) |
| White women (n = 19) | 1 (5%) | 10 (53%) | 8 (42%) |
| LUNG CANCER | | | |
| Black men (n = 20) | 8 (40%) | 5 (25%) | 7 (35%) |
| Black women (n = 16) | 0 (0%) | 6 (38%) | 10 (63%) |
| White men (n = 20) | 2 (10%) | 13 (65%) | 5 (25%) |
| White women (n = 11) | 2 (18%) | 4 (36%) | 5 (45%) |
| PROSTATE CANCER | | | |
| Black men (n = 20) | 0 (0%) | 9 (45%) | 11 (55%) |
| White men (n = 19) | 2 (11%) | 13 (68%) | 4 (21%) |
| NIDDM | | | |
| Black men (n = 2) | 0 (0%) | 1 (50%) | 1 (50%) |
| Black women (n = 3) | 1 (33%) | 1 (33%) | 1 (33%) |
| White men (n = 4) | 0 (0%) | 0 (0%) | 4 (100%) |
| White women (n = 9) | 6 (67%) | 2 (22%) | 1 (11%) |
| ESRD due to NIDDM | | | |
| Black men (n = 6) | 0 (0%) | 1 (17%) | 5 (83%) |
| Black women (n = 8) | 0 (0%) | 2 (25%) | 6 (75%) |
| White men (n = 5) | 0 (0%) | 2 (40%) | 3 (60%) |
| White women (n = 4) | 0 (0%) | 2 (50%) | 2 (50%) |
| HYPERTENSION (HTN) | | | |
| Black men (n = 12) | 0 (0%) | 3 (25%) | 9 (75%) |
| Black women (n = 14) | 0 (0%) | 2 (17%) | 12 (83%) |
| White men (n = 11) | 1 (9%) | 5 (45%) | 5 (45%) |
| White women (n = 10) | 1 (10%) | 6 (60%) | 3 (30%) |
| ESRD due to HTN | | | |
| Black men (n = 10) | 1 (10%) | 2 (20%) | 7 (70%) |
| Black women (n = 9) | 0 (0%) | 0 (0%) | 9 (100%) |
| White men (n = 9) | 0 (0%) | 5 (56%) | 4 (44%) |
| White women (n = 9) | 0 (0%) | 3 (33%) | 6 (67%) |
| MYOCARDIAL INFARCTION | | | |
| White women (n = 7) | 0 (0%) | 5 (71%) | 2 (29%) |

In Table 8, the susceptibility allele is indicated, as well as the odds ratio (OR). Haldane's correction was used if the denominator was zero. If the odds ratio (OR) was ≧1.5, the 95% confidence interval (C.I.) is also given. An odds ratio of 1.5 was chosen as the threshold of significance based on the recommendation of Austin et al. in Epidemiol Rev., 16:65-76, (1994). Odds high-lighted below.

TABLE 8
ALLELE-SPECIFIC ODDS RATIOS

| | SUSCEPTIBILITY | | |
|---|---|---|---|
| DISEASE | ALLELE | OR | 95% C.I. |
| Breast Cancer | | | |
| Black women | T | 1.5 | 0.6-4.0 |
| White women | C | 1.0 | |
| Lung Cancer | | | |
| Black men | C | 8.2 | 3.3-20 |
| Black women | T | 1.4 | |
| White men | T | 0.8 | |
| White women | C | 1.3 | |
| Prostate Cancer | | | |
| Black men | C | 2.1 | 0.8-5.8 |
| White men | C | 0.8 | |
| NIDDM | | | |
| Black men | C | 2.5 | 0.2-26 |
| Black women | C | 3.1 | 0.6-17 |
| White men | T | 10.6 | 1.4-81 |
| White women | C | 7.8 | 2.4-26 |
| ESRD due to NIDDM* | | | |
| Black men | T | 3.7 | 0.2-78 |
| Black women | T | 7.0 | 0.8-62 |
| White men | C | 5.0 | 0.5-47 |
| White women | T | 10.5 | 1.5-74 |
| Hypertension (HTN) | | | |
| Black men | C | 1.1 | |
| Black women | T | 3.5 | 0.8-17 |
| White men | T | 1.3 | |
| White women | C | 1.5 | 0.6-40 |
| ESRD due to HTN*¹ | | | |
| Black men | C | 1.8 | 0.3-9.0 |
| Black women | T | 4.1 | 0.5-37 |
| White men | T | 1.2 | |
| White women | T | 2.3 | 0.5-11 |
| Myocardial Infarction | | | |
| White women | C | 1.2 | |

*Compared to group with NIDDM alone.
*¹Compared to group with HTN alone.

Genotype-Specific Odds Ratios

In Table 9, the susceptibility allele (S) is indicated, and the alternative allele at this locus is defined as the protective allele (P). Also presented is the odds ratio (OR) for the SS and SP genotypes. The odds ratio for the PP genotype is 1 by definition, since it is the reference group, and is not presented in the table below. For odds ratios ≧1.5, the asymptotic 95% confidence interval (C.I.) is also given, in parentheses. An odds ratio of 1.5 was chosen as the threshold of significance based on the recommendation of Austin et al., in Epidemiol. Rev., 16:65-76 (1994).

Odds ratios of 1.5 or higher are high-lighted below. Haldane's correction was used when the denominator was zero. To minimize confusion, genotype-specific odds ratios are presented only for diseases in which the allele-specific odds ratio was at least 1.5.

TABLE 9
GENOTYPE-SPECIFIC ODDS RATIOS

| | SUSCEPTI- | | |
|---|---|---|---|
| DISEASE | BILITY ALLELE | OR(SS) | OR(SP) |
| Breast Cancer | | | |
| Black women | T | 3.1 (0.3-28) | 2.6 (0.3-24) |
| Lung Cancer | | | |
| Black men | C | 74 (9.1-598) | 2.3 (0.9-5.7) |
| Prostate Cancer | | | |
| Black men | C | 2.8 (0.2-47) | 2.6 (1.2-5.6) |
| NIDDM | | | |
| Black men | C | 22 (1.1-437) | 3.1 (0.6-17) |
| Black women | C | 11 (0.5-240) | 1.5 (0.1-26) |
| White men | T | 3.4 (0.4-30) | 0.3 |
| White women | C | 60 (4.6-782) | 1.6 (0.1-19) |
| ESRD due to | | | |
| NIDDM* | | | |
| Black men | T | 3.7 (0.2-78) | 1.0 |
| Black women | T | 13 (1.0-173) | 5.0 (0.3-73) |
| White men | C | 1.3 | 6.4 (0.6-68) |
| White women | T | 22 (1.8-261) | 13 (1.2-141) |
| Hypertension (HTN) | | | |
| Black women | T | 2.9 (0.3-26) | 0.9 |
| White women | C | 3.3 (0.2-49) | 1.6 (0.4-7.2) |
| ESRD due to | | | |
| HTN*¹ | | | |
| Black men | C | 3.8 (0.4-40) | 0.9 |
| Black women | T | 0.8 | 0.2 |
| White women | T | 5.6 (0.5-64) | 1.6 (0.1-19) |

*Compared to group with NIDDM alone.
*¹Compared to group with HTN alone.

The control samples agree with Hardy-Weinberg equilibrium, as follows:

A frequency of 0.12 for the C allele (“p”) and 0.88 for the T allele (“q”) among black male control individuals predicts genotype frequencies of 1% C/C, 22% C/T, and 77% T/T at Hardy-Weinberg equilibrium (p²+2pq+q²=1). The observed genotype frequencies were 0% C/C, 24% C/T, and 76% T/T, in excellent agreement with those predicted for Hardy-Weinberg equilibrium.

A frequency of 0.24 for the C allele (“p”) and 0.76 for the T allele (“q”) among black female control individuals predicts genotype frequencies of 6% C/C, 36% C/T, and 58% T/T at Hardy-Weinberg equilibrium (p²+2pq+q²=1). The observed genotype frequencies were 5% C/C, 38% C/T, and 57% T/T, in excellent agreement with those predicted for Hardy-Weinberg equilibrium.

A frequency of 0.38 for the C allele (“p”) and 0.62 for the T allele (“q”) among white male control individuals predicts genotype frequencies of 14% C/C, 48% C/T, and 38% T/T at Hardy-Weinberg equilibrium (p²+2pq+q²=1). The observed genotype frequencies were 13% C/C, 50% C/T, and 37% T/T, in excellent agreement with those predicted for Hardy-Weinberg equilibrium.

A frequency of 0.31 for the C allele (“p”) and 0.69 for the T allele (“q”) among white female control individuals predicts genotype frequencies of 10% C/C, 42% C/T, and 48% T/T at Hardy-Weinberg equilibrium (p²+2pq+q²=1). The observed genotype frequencies were 4% C/C, 53% C/T, and 43% T/T, in fair agreement with those predicted for Hardy-Weinberg equilibrium.

Using an allele-specific odds ratio of 1.5 or greater as a practical level of significance, the following observations can be made.

Among black women with breast cancer, the odds ratio for the T allele at this locus was 1.5 (95% CI, 0.6-4.0). The odds ratio for the TC heterozygote was 2.6 (95% CI, 0.3-24), and 3.1 (95% CI, 0.3-28) for the TT homozygote. The genotype-specific odds ratios suggest that the T allele behaves as a dominant susceptibility allele.

For black men with lung cancer, the odds ratio for the C allele at this locus was 8.2 (95% CI, 3.3-20). The odds ratio for the CT heterozygote was 2.3 (95% CI, 0.9-5.7), and 74 (95% CI, 9.1-598) for the CC homozygote. The genotype-specific odds ratios suggest that the T allele behaves as a dominant susceptibility allele, since the heterozygote (with one allele copy) has an odds ratio of 2.3. However, there is a pronounced (more than multiplicative) effect of gene dosage, since the homozygote with two copies of the C allele displayed a more than 30-fold larger odds ratio.

For black men with prostate cancer, the odds ratio for the C allele at this locus was 2.1 (95% CI, 0.8-5.8). The odds ratio for the heterozygote (2.6, 95% CI, 1.2-5.6) was essentially the same as for the CC homozygote (2.8, 95% CI, 0.2-47), suggesting that the C allele behaves in a dominant fashion.

For black men with NIDDM, the odds ratio for the C allele at this locus was 2.5 (95% CI, 0.2-26). The odds ratio for the heterozygote was 3.1 (95% CI, 0.6-17), and for the CC homozygote was 22 (95% CI, 1.1-437). The genotype-specific odds ratios suggest that the C allele behaves as a dominant susceptibility allele, since the heterozygote (with one allele copy) had an odds ratio of 3.1. However, there was a pronounced effect of gene dosage, since the homozygote with two copies of the C allele displayed a more than 7-fold larger odds ratio than the heterozygote.

For black women with NIDDM, the odds ratio for the C allele at this locus was 3.1 (95% CI, 0.6-17). The odds ratio for the heterozygote was 1.5 (95% CI, 0.1-26), and for the CC homozygote was 11 (95% CI, 0.5-240). The genotype-specific odds ratios suggest that the C allele behaves as a dominant susceptibility allele, since the heterozygote (with one allele copy) had an odds ratio of 1.5. However, there is a pronounced (more than multiplicative) effect of gene dosage, since the homozygote with two copies of the C allele displayed a more than 7-fold larger odds ratio than the heterozygote.

For white men with NIDDM, the odds ratio for the T allele at this locus was 10.6 (95% CI, 1.4-81). The odds ratio for the heterozygote was actually less than one (0.3), but for the TT homozygote was 3.4 (95% CI, 0.4-30). The genotype-specific odds ratios suggest that the T allele behaves as a recessive susceptibility allele.

For white women with NIDDM, the odds ratio for the C allele at this locus was 7.8 (95% CI, 2.4-26). The odds ratio for the heterozygote was 1.6 (95% CI, 0.1-19), and for the CC homozygote was 60 (95% CI, 4.6-782). The genotype-specific odds ratios suggest that the C allele behaves as a dominant susceptibility allele, since the heterozygote (with one allele copy) had an odds ratio of 1.6. However, there is a pronounced (more than multiplicative) effect of gene dosage, since the homozygote with two copies of the C allele displayed a more than 37-fold larger odds ratio than the heterozygote.

For black men with ESRD due to NIDDM, the odds ratio for the T allele at this locus was 3.7 (95% CI, 0.2-78), compared with black men with NIDDM but no renal disease. The odds ratio for the heterozygote was 1.0, but for the TT homozygote was 3.7 (95% CI, 0.2-78). The genotype-specific odds ratios suggest that the T allele behaves as a recessive susceptibility allele.

For black women with ESRD due to NIDDM, the odds ratio for the T allele at this locus was 7.0 (95% CI, 0.8-62), compared with black women with NIDDM but no renal disease. The odds ratio for the heterozygote was 5.0 (95% CI, 0.3-73), and for the TT homozygote was 13 (95% CI, 1.0-173). The genotype-specific odds ratios suggest that the T allele behaves as a dominant susceptibility allele. However, there is a pronounced (more than additive) effect of gene dosage, since the homozygote with two copies of the C allele displayed a more than two-fold larger odds ratio than the heterozygote.

For white men with ESRD due to NIDDM, the odds ratio for the C allele at this locus was 5.0 (95% CI, 0.5-47) vs. white men with NIDDM but no renal disease. Inspection of the genotype-specific odds ratios suggests that the C allele is codominant, since the heterozygote had a much higher odds ratio (6.4, 95% CI 0.6-68) than the CC homozygote (1.3) or the reference TT genotype (odds ratio 1, by definition).

For white women with ESRD due to NIDDM, the odds ratio for the T allele at this locus was 10.5 (95% CI, 1.5-74) vs. white women with NIDDM but no renal disease. The odds ratio for the heterozygote was 13 (95% CI, 1.2-141), and the TT homozygote was 22 (95% CI, 1.8-261). The genotype-specific odds ratios suggest that the T allele behaves as a dominant susceptibility allele. However, there is a pronounced (approximately additive) effect of gene dosage, since the homozygote with two copies of the T allele displayed a roughly two-fold larger odds ratio than the heterozygote.

For black women with hypertension, the odds ratio for the T allele at this locus was 3.5 (95% CI, 0.8-17). The odds ratio for the heterozygote was 0.9, but for the TT homozygote was 2.9 (95% CI, 0.3-26). The genotype-specific odds ratios suggest that the T allele behaves as a recessive susceptibility allele.

For white women with hypertension, the odds ratio for the C allele at this locus was 1.5 (95% CI, 0.6-40). The odds ratio for the heterozygote was 1.6 (95% CI, 0.4-7.2), and for the CC homozygote was 3.3 (95% CI, 0.2-49). The genotype-specific odds ratios suggest that the C allele behaves in a dominant fashion, with a strictly additive effect of allele dosage, since 1.6+1.6˜3.3.

For black men with ESRD due to hypertension (HTN), the odds ratio for the C allele at this locus was 1.8 (95% CI, 0.3-9.0) relative to black men with HTN but no renal failure. The odds ratio for the heterozygote was 0.9, but for the CC homozygote was 3.8 (95% CI, 0.4-40). The genotype-specific odds ratios suggest that the C allele behaves in a recessive fashion.

For black women with ESRD due to HTN, the odds ratio for the T allele was 4.1 (95% CI, 0.5-37) relative to black women with HTN alone. The genotype-specific odds ratios were found to be unhelpful, so no inference can be drawn about whether the T allele behaves in a dominant, recessive, or codominant fashion.

For white women with ESRD due to HTN, the odds ratio for the T allele was 2.3 (95% CI, 0.5-11) relative to white women with HTN alone. The odds ratio for the heterozygote was 1.6 (95% CI, 0.1-19), and for the TT homozygote was 5.6 (95% CI, 0.5-64). The genotype-specific odds ratios suggest that the C allele behaves in a dominant fashion, with a more than multiplicative effect of allele dosage, since 5.6/(1.6)²=5.6/3.56=1.6>1.

According to commercially available software [GENOMATIX MatInspector Professional; http://genomatix.qsf.de/cqi-bin/matinspector/matinspector.pl; Quandt et al., Nucleic Acids Res. 23: 4878-4884 (1995)], the C2684→T SNP is predicted to have the following potential effects on transcription of the ecNOS gene:

a. Disruption of an NF1 (nuclear factor 1) binding site, which consists of the sequence 5′-CCCTGGCCGGCTGACCCT-3′ (SEQ ID NO: 9), beginning at position +2677 on the (+) strand. This polymorphism replaces the indicated C with a T, which should result in a weaker binding site for NF1, a transcriptional activator of ecNOS. NF1 binding sites occur rather frequently, 4.11 times per 1000 base pairs of random genomic sequence. Since NF-1 is a positive transcriptional regulator, disruption of its binding site is expected to result in a decreased rate of transcription of the ecNOS gene. If the rate of translation is tied to the level of messenger RNA, as is the case for most proteins, then less gene product (ecNOS enzyme) will be the result, ultimately leading to less nitric oxide (NO) produced in tissues such as endothelial cells.

b. Disruption of an ER (estrogen receptor) binding site, which consists of the sequence 5′-CCCTGGCCGGCTGACCCT-3′ (SEQ ID NO: 9), beginning at position +2677 on the (+) strand. This polymorphism replaces the indicated C with a T, which should result in a weaker binding site for the estrogen receptor, a transcriptional activator of ecNOS. ER binding sites occur moderately frequently, at the rate of 1.73 sites per 1000 base pairs of random genomic sequence. Since the estrogen receptor is a transcriptional activator, disruption of its binding site is expected to result in a decreased rate of transcription of the ecNOS gene. If the rate of translation is tied to the level of messenger RNA, as is the case for most proteins, then less gene product (ecNOS enzyme) will be the result, ultimately leading to less nitric oxide (NO) produced in tissues such as endothelial cells. In rodents, androgens have been shown to accelerate renal failure. Thus, it is intriguing that this polymorphism might interfere with the effect of estrogen, essentially tilting the balance towards androgens.

c. Disruption of a TCF11 (TCF11/KCR-F1/Nrf1 homodimer) binding site, which consists of the sequence 5′-GTCAGCCGGCCAG-3′ (SEQ ID NO: 10), which ends at position +2679 on the (−) strand. This polymorphism replaces the C on the (+) strand by a T on the (+) strand. The complementary base on the (−) strand is thus changed from the reference sequence G, indicated in TCF11's binding site, above, to an A, complementary to the T of the polymorphism. The TCF11 binding site occurs rather frequently, at the rate of 4.63 times per 1000 base pairs of random genomic sequence. Involvement of the TCF11 homodimer in regulation of ecNOS has not previously been demonstrated.

d. Disruption of an AP4 (activator protein 4) binding site, which consists of the sequence 5′-GTCAGCCGGC-3′ (SEQ ID NO: 11), which ends at position +2682 on the (−) strand. The C2684→T polymorphism replaces the C on the (+) strand by a T on the (+) strand. The complementary base on the (−) strand thus becomes A, rather than the reference sequence G, as indicated immediately above. AP4 is a potent transcriptional activator. Its sites occur with only moderate frequency in genomic DNA: 0.96 times per 1000 base pairs in a random genomic sequence of vertebrates. Disruption of an AP4 site is predicted to lead to a decrease in transcription of the ecNOS gene, with a resultant decrease in tissue nitric oxide production.

e. Disruption of a VMAF (v-Maf) binding site, which consists of the sequence 5′-GCCGGCTGACCCTGCCTCA-3′ (SEQ ID NO: 12), beginning at position +2682 on the (+) strand. Thus, the C2684→T polymorphism replaces the indicated C by a T. VMAF sites occur moderately frequently, i.e., 0.99 times per 1000 base pairs of random genomic sequence in vertebrates. At the moment, very little is known about the regulation of ecNOS by the cellular homolog of v-Maf.

Sim et al., Mol. Genet. Metab., 65: 562 (1998), reported a disruption of a MspI restriction site in the ecNOS gene. However, the specific MspI site reported in Sim et al., was not further identified by sequencing, and there are 11 MspI restriction sites predicted in the sequence we have examined (GenBank Accession Number AF032908).

### Example 3

### C to T Transition at Position 2575

Methods of DNA amplification, sequencing and data analysis were essentially as described in Example 1 except that the forward primer was 5′ gagtctggccaacacaaatcc 3′ (SEQ ID NO: 13) and the reverse primer was 5′ ctctagggtcatgcaggttctc 3′ (SEQ ID NO: 14). A substitution mutation (transition) was found in which the C found in the reference sequence (SEQ ID NO: 1) was replaced with a T. Data analysis produced the following results.

TABLE 9
ALLELE FREQUENCIES

| | |
|---|---|
| C | T |

| | | | |
|---|---|---|---|
| CONTROL | | | |
| Black men (n = 64 chromosomes) | 61 | (95%) | 3 (5%) |
| Black women (n = 70 chromosomes) | 70 | (100%) | 0 (0%) |
| White men (n = 84 chromosomes) | 84 | (100%) | 0 (0%) |
| White women (n = 102 chromosomes) | 102 | (100%) | 0 (0%) |
| DISEASE | | | |
| BREAST CANCER | | | |
| Black women (n = 40 chromosomes) | 38 | (95%) | 2 (5%) |
| White women (n = 38 chromosomes) | 38 | (100%) | 0 (0%) |
| LUNG CANCER | | | |
| Black men (n = 38 chromosomes) | 38 | (100%) | 0 (0%) |
| Black women (n = 32 chromosomes) | 30 | (94%) | 2 (6%) |
| White men (n = 40 chromosomes) | 40 | (100%) | 0 (0%) |
| White women (n = 22 chromosomes) | 22 | (100%) | 0 (0%) |
| PROSTATE CANCER | | | |
| Black men (n = 40 chromosomes) | 39 | (98%) | 1 (3%) |
| White men (n = 40 chromosomes) | 40 | (100%) | 0 (0%) |
| NIDDM | | | |
| Black men (n = 4 chromosomes) | 4 | (100%) | 0 (0%) |
| Black women (n = 8 chromosomes) | 8 | (100%) | 0 (0%) |
| White men (n = 8 chromosomes) | 8 | (100%) | 0 (0%) |
| White women (n = 6 chromosomes) | 6 | (100%) | 0 (0%) |
| ESRD DUE TO NIDDM | | | |
| Black men (n = 12 chromosomes) | 12 | (100%) | 0 (0%) |
| Black women (n = 16 chromosomes) | 16 | (100%) | 0 (0%) |
| White men (n = 10 chromosomes) | 10 | (100%) | 0 (0%) |
| White women (n = 8 chromosomes) | 8 | (100%) | 0 (0%) |
| HYPERTENSION (HTN) | | | |
| Black men (n = 22 chromosomes) | 21 | (95%) | 1 (5%) |
| Black women (n = 16 chromosomes) | 12 | (75%) | 4 (25%) |
| White men (n = 20 chromosomes) | 20 | (100%) | 0 (0%) |
| White women (n = 18 chromosomes) | 18 | (100%) | 0 (0%) |
| ESRD DUE TO HTN | | | |
| Black men (n = 14 chromosomes) | 14 | (100%) | 0 (0%) |
| Black women (n = 12 chromosomes) | 12 | (100%) | 0 (0%) |
| White men (n = 14 chromosomes) | 14 | (100%) | 0 (0%) |
| White women (n = 8 chromosomes) | 8 | (100%) | 0 (0%) |
| MYOCARDIAL INFARCTION | | | |
| White women (n = 14 chromosomes) | 14 | (100%) | 0 (0%) |

TABLE 10
GENOTYPE FREQUENCIES

| | C/C | C/T | T/T |
|---|---|---|---|
| CONTROLS | | | |
| Black men (n = 32) | 29 (91%) | 3 (9%) | 0 (0%) |
| Black women (n = 35) | 35 (100%) | 0 (0%) | 0 (0%) |
| White men (n = 42) | 42 (100%) | 0 (0%) | 0 (0%) |
| White women (n = 51) | 51 (100%) | 0 (0%) | 0 (0%) |
| DISEASE | | | |
| BREAST CANCER | | | |
| Black women (n = 20) | 18 (90%) | 2 (10%) | 0 (0%) |
| White women (n = 19) | 19 (100%) | 0 (0%) | 0 (0%) |
| LUNG CANCER | | | |
| Black men (n = 19) | 19 (100%) | 0 (0%) | 0 (0%) |
| Black women (n = 16) | 14 (88%) | 2 (13%) | 0 (0%) |
| White men (n = 20) | 20 (100%) | 0 (0%) | 0 (0%) |
| White women (n = 11) | 11 (100%) | 0 (0%) | 0 (0%) |
| PROSTATE CANCER | | | |
| Black men (n = 20) | 19 (95%) | 1 (5%) | 0 (0%) |
| White men (n = 20) | 20 (100%) | 0 (0%) | 0 (0%) |
| NIDDM | | | |
| Black men (n = 2) | 2 (100%) | 0 (0%) | 0 (0%) |
| Black women (n = 4) | 4 (100%) | 0 (0%) | 0 (0%) |
| White men (n = 4) | 4 (100%) | 0 (0%) | 0 (0%) |
| White women (n = 3) | 3 (100%) | 0 (0%) | 0 (0%) |
| ESRD due to NIDDM | | | |
| Black men (n = 6) | 6 (100%) | 0 (0%) | 0 (0%) |
| Black women (n = 8) | 8 (100%) | 0 (0%) | 0 (0%) |
| White men (n = 5) | 5 (100%) | 0 (0%) | 0 (0%) |
| White women (n = 4) | 4 (100%) | 0 (0%) | 0 (0%) |
| HYPERTENSION (HTN) | | | |
| Black men (n = 11) | 10 (91%) | 1 (9%) | 0 (0%) |
| Black women (n = 8) | 4 (50%) | 4 (50%) | 0 (0%) |
| White men (n = 10) | 10 (100%) | 0 (0%) | 0 (0%) |
| White women (n = 9) | 9 (100%) | 0 (0%) | 0 (0%) |
| ESRD due to HTN | | | |
| Black men (n = 7) | 7 (100%) | 0 (0%) | 0 (0%) |
| Black women (n = 6) | 6 (100%) | 0 (0%) | 0 (0%) |
| White men (n = 7) | 7 (100%) | 0 (0%) | 0 (0%) |
| White women (n = 4) | 4 (100%) | 0 (0%) | 0 (0%) |
| MYOCARDIAL INFARCTION | | | |
| White women (n = 7) | 7 (100%) | 0 (0%) | 0 (0%) |

Allele-Specific Odds Ratios

The susceptibility allele is indicated, as well as the odds ratio (OR). Haldane's correction was used if the denominator was zero. If the odds ratio (OR) is ≧1.5, the 95% confidence interval (C.I.) is also given. An odds ratio of 1.5 was chosen as the threshold of significance based on the recommendation of Austin et al., in Epidemiol. Rev., 16:65-76, (1994). “[E]pidemiology in general and case-control studies in particular are not well suited for detecting weak associations (odds ratios <1.5).” Id. at 66. Odds ratios of 1.5 or higher are high-lighted below.

TABLE 11
ALLELE-SPECIFIC ODDS RATIOS

| | SUSCEPTIBILITY | | |
|---|---|---|---|
| DISEASE | ALLELE | OR | 95% C.I. |
| Breast Cancer | | | |
| Black women | T | 9.2 | 1.1-80 |
| White women | C | 1.0 | |
| Lung Cancer | | | |
| Black men | C | 4.4 | 0.5-36 |
| Black women | T | 11.6 | 1.3-101 |
| White men | C | 1.0 | |
| White women | C | 1.0 | |
| Prostate Cancer | | | |
| Black men | C | 1.9 | 0.2-19 |
| White men | C | 1.0 | |
| NIDDM | | | |
| Black men | C | 2.0 | 0.2-18 |
| Black women | C | 1.0 | |
| White men | C | 1.0 | |
| White women | C | 1.0 | |
| ESRD due to NIDDM* | | | |
| Black men | C | 1.0 | |
| Black women | C | 1.0 | |
| White men | C | 1.0 | |
| White women | C | 1.0 | |
| Hypertension (HTN) | | | |
| Black men | C | 0.8 | |
| Black women | T | 50.8 | 6.2-418 |
| White men | C | 1.0 | |
| White women | C | 1.0 | |
| ESRD due to HTN*¹ | | | |
| Black men | C | 2.0 | 0.2-20 |
| Black women | C | 9.0 | 1.1-76 |
| White men | C | 1.0 | |
| White women | C | 1.0 | |
| Myocardial Infarction | | | |
| White women | C | 1.0 | |

*Compared to group with NIDDM alone.
*¹Compared to group with HTN alone.

Genotype-Specific Odds Ratios

In Table 12, the susceptibility allele (S) is indicated; the alternative allele at this locus is defined as the protective allele (P). Also presented is the odds ratio (OR) for the SS and SP genotypes. The odds ratio for the PP genotype is 1 by definition, since it is the reference group, and is not presented in the table below. For odds ratios ≧1.5, the asymptotic 95% confidence interval (C.I.) is also given, in parentheses.

Odds ratios of 1.5 or higher are high-lighted below. Haldane's correction was used when the denominator was zero. To minimize confusion, genotype-specific odds ratios are presented only for diseases in which the allele-specific odds ratio was at least 1.5.

TABLE 12
GENOTYPE-SPECIFIC ODDS RATIOS

| | SUSCEPTI- | | | |
|---|---|---|---|---|
| DISEASE | BILITY ALLELE | OR(SS) | OR(SP) | |
| Breast | | | | |
| Cancer | | | | |
| Black | T | 1.9 (0.1-32) | 9.6 | (1.1-85) |
| women | | | | |
| Lung Cancer | | | | |
| Black men | T* | 1.5 (0.1-25) | 0.2 | (0-1.8) |
| Black | T | 2.4 (0.1-41) | 12.2 | (1.4-109.0) |
| women | | | | |
| Prostate | | | | |
| Cancer | | | | |
| Black men | T* | 1.5 (0.1-25) | 0.6 | (0.2-2.7) |
| NIDDM | | | | |
| Black men | T | 11.8 (0.6-218) | 1.7 | (0.2-17) |
| Hypertension | | | | |
| (HTN) | | | | |
| Black | T | 7.9 (0.5-137) | 71 | (8.0-628) |
| women | | | | |
| ESRD due to | | | | |
| HTN | | | | |
| Black men*¹ | T* | 3.9 (0.2-67) | 0.6 | (0.1-4.9) |
| Black | T* | 5.5 (0.3-93) | 5.5 | (0.3-93) |
| women*¹ | | | | |

*C, not T, is the susceptibility allele according to the allele-specific odds ratio (see table above).
*¹Compared to group with HTN alone.

The control samples agree with Hardy-Weinberg equilibrium, as follows:

A frequency of 0.95 for the C allele (“p”) and 0.05 for the T allele (“q”) among black male control individuals predicts genotype frequencies of 90% C/C, 10% C/T, and 0% T/T at Hardy-Weinberg equilibrium (p²+2pq+q²=1). The observed genotype frequencies were 91% C/C, 9% C/T, and 0% T/T, in excellent agreement with those predicted for Hardy-Weinberg equilibrium.

A frequency of 1.0 for the C allele (“p”) and 0 for the T allele (“q”) among black female control individuals predicts genotype frequencies of 100% C/C, 0% C/T, and 0% T/T at Hardy-Weinberg equilibrium (p²+2pq+q²=1). The observed genotype frequencies were 100% C/C, 0% C/T, and 0% T/T, in perfect agreement with those predicted for Hardy-Weinberg equilibrium.

A frequency of 1.0 for the C allele (“p”) and 0 for the T allele (“q”) among white male control individuals predicts genotype frequencies of 100% C/C, 0% C/T, and 0% T/T at Hardy-Weinberg equilibrium (p²+2pq+q²=1). The observed genotype frequencies were 100% C/C, 0% C/T, and 0% T/T, in perfect agreement with those predicted for Hardy-Weinberg equilibrium.

A frequency of 1.0 for the C allele (“p”) and 0 for the T allele (“q”) among white female control individuals predicts genotype frequencies of 100% C/C, 0% C/T, and 0% T/T at Hardy-Weinberg equilibrium (p²+2pq+q²=1). The observed genotype frequencies were 100% C/C, 0% C/T, and 0% T/T, in perfect agreement with those predicted for Hardy-Weinberg equilibrium.

Using an allele-specific odds ratio of 1.5 or greater as a practical level of significance, the following observations can be made.

Among black women with breast cancer, the odds ratio for the T allele at this locus was 9.2 (95% CI, 1.1-80). The odds ratio for the TC heterozygote was 9.6 (95% CI, 1.1-85), considerably higher than for the TT homozygote, which was 1.9 (95% CI, 0.1-32). When the heterozygote has a different odds ratio than either homozygote, the alleles are said to be codominant (Khoury et al., Fundamentals of Genetic Epidemiology, Oxford University Press: 33 (1993)).

For black men with lung cancer, the odds ratio for the C allele at this locus was 4.4 (95% CI, 0.5-36). However, in this case the genotype-specific odds ratios were unhelpful in suggesting whether the C allele functions as a recessive, dominant, or codominant allele because the C allele no longer appears as the susceptibility allele.

For black women with lung cancer, the odds ratio for the T allele at this locus was 11.6 (1.3-101). Inspection of the genotype-specific odds ratios suggests that the T allele is codominant, since the heterozygote has a much higher odds ratio (12.2, 95% CI 1.4-109) than the TT homozygote (2.4, 95% CI 0.1-41) or the reference CC genotype (odds ratio 1, by definition).

For black men with prostate cancer, the odds ratio for the C allele at this locus was 1.9 (95% CI, 0.2-19). However, in this case the genotype-specific odds ratios are unhelpful in suggesting whether the C allele functions as a recessive, dominant, or codominant allele because the C allele no longer appears as the susceptibility allele.

For black men with NIDDM, the odds ratio for the C allele at this locus was 2.0 (95% CI, 0.2-18). However, in this case the genotype-specific odds ratios are again unhelpful in suggesting whether the C allele functions as a recessive, dominant, or codominant allele because the C allele no longer appears as the susceptibility allele.

For black women with hypertension (HTN), the odds ratio for the T allele at this locus was 50.8 (95% CI, 6.2-418). Inspection of the genotype-specific odds ratios suggests that the T allele is codominant, since the heterozygote had a much higher odds ratio (71, 95% CI 8.0-628) than the TT homozygote (7.9, 95% CI, 0.5-137) or the reference CC genotype (odds ratio 1, by definition).

For black men with ESRD due to hypertension (HTN), the odds ratio for the C allele at this locus was 2.0 (95% CI, 0.2-20) when compared with black men with HTN. However, in this case the genotype-specific odds ratios were unhelpful in suggesting whether the C allele functions as a recessive, dominant, or codominant allele because the C allele no longer appears as the susceptibility allele.

For black women with ESRD due to hypertension (HTN), the odds ratio for the C allele at this locus was 9.0 (95% CI, 1.1-76) when compared with black women with HTN. However, in this case the genotype-specific odds ratios were unhelpful in suggesting whether the C allele functions as a recessive, dominant, or codominant allele because the C allele no longer appears as the susceptibility allele.

According to commercially available software [GENOMATIX MatInspector Professional; http://genomatix.gsf.de/cgi-bin/matinspector/matinspector.pl; Quandt et al., Nucleic Acids Res. 23: 4878-4884 (1995)], the G2458→A SNP is predicted to have the following potential effects on transcription of the ecNOS gene:

a. Disruption of a STAF_{—}01 (Se-Cys tRNA gene transcription activating factor 1) site (5′-AAACCCCAGCATGCACTCTGGC-3′ (SEQ ID NO: 15) beginning at position 2560 on the (+) strand. This polymorphism results in replacement of the indicated C by a T. STAF_{—}01 sites occur extremely rarely in the genome: 0.02 occasions per 1000 base pairs of random genomic sequence in vertebrates.

STAF is a transcriptional activator possessing seven zinc finger domains. It belongs to a family of similar transcription factors (Myslinski et al., J. Biol. Chem., 273(34):21998-22006, 1998). Although originally described as an activator of transcription by RNA polymerase III from the selenocysteine tRNA gene in Xenopus and the mouse, and by RNA polymerase II from small nuclear RNA-type genes such as U6 snRNA in humans, STAF can also activate transcription of other genes by RNA polymerase II (Schuster et al., Mol. Cell Biol., 18(5):2650-2658, 1998).

Since STAF is a positive transcriptional regulator, disruption of its binding site is expected to result in a decreased rate of transcription of the ecNOS gene. If the rate of translation is tied to the level of messenger RNA, as is the case for many proteins, then the T allele is expected to result in less gene product (ecNOS enzyme), ultimately leading to less nitric oxide (NO) produced in tissues such as endothelial cells.

b. Disruption of a TH1E47_{—}01 (Thing1/E47 heterodimer) site. Thing1 is also called Hxt, eHAND, or Hand1 (Scott et al., Mol. Cell. Biol., 20(2):530-541, 2000). The putative binding site for the heterodimer (5′-CATGCACTCTGGCCTG-3′ (SEQ ID NO: 16) begins at position +2569 on the (+) strand. This polymorphism results in replacement of the indicated C by a T. TH1E47_{—}01 sites occur relatively often in the genome: 2.04 occasions per 1000 base pairs of random genomic sequence in vertebrates.

E47 usually functions as a transcriptional activator. Binding of E47 by Thing1/Hxt/eHAND/Hand1, which itself can be a transcriptional activator for trophoblast during development (Scott et al., op. cit.), may actually result in repression of E47's activity. As a further complication to predicting the nature of TH1E47's effect on the ecNOS gene, whether positive or negative, activity of the E47 homodimer is repressed by phosphorylation (Neufeld B et al., J. Biol. Chem., 275(27): 20239-42, 2000). Phosphorylation has not yet been reported to affect the activity of the Hand1/E47 heterodimer.

c. Disruption of an NF1_Q6 (nuclear factor 1) site (5′-CTCTGGCCTGAAGTGCCT-3′ (SEQ ID NO: 17) beginning at position +2575 on the (+) strand. This polymorphism results in replacement of the indicated C by a T. NF1_Q6 sites occur relatively frequently in the genome: 4.11 sites per 1000 base pairs of random genomic sequence in vertebrates. NF1, usually a transcriptional activator, has not yet been shown to affect expression of the ecNOS gene.

### EXAMPLE 4

### Deletion at Position 1272

Sample collection and DNA isolation were as described in Example 1.

DNA Amplification

DNA encoding the eNOS promoter region was amplified by polymerase chain reaction (PCR). One hundred nanograms of purified genomic DNA was used in each PCR reaction. The forward primer was 5′agcagtgcaccaaggaaaatgagg 3′ (SEQ ID NO: 18) and the reverse primer was 5′ agtgcagtggtgtgatcttggttc 3′ (SEQ ID NO: 19). The reaction mix consisted of 100 ng leukocyte genomic DNA, 10 pmol of each primer, 200 nM dNTPs, 1 U Taq DNA polymerase (Perkin-Elmer), 1×PCR buffer (50 mM KCl, 10 mM Tris-HCl, pH 8.3, 1.5 mM MgCl₂, and 0.01% [w/v] gelatin) and 3% (v/v) DMSO. The total reaction volume was 25 μl. The PCR protocol used consisted of 4 minutes at 95° C. followed by 29 cycles of a 40 second denaturation step at 95° C., a 20 second annealing step at 59° C. and a 1 minute extension step at 73° C. After the completion of the 29 cycles a final extension reaction was conducted at 73° C. for 4 minutes. The PCR product obtained was then purified using QIAquick 96 PCR purification kit (Qiagen, Inc. Valencia, Calif.) following the manufacturer's protocol. Purified PCR product was then used for sequencing.

DNA Sequencing

Purified PCR product was sequenced by cycle sequencing using a Perkin-Elmer dye terminator kit according to the manufacturer's protocol Briefly, 8 μl of terminator ready reaction mix (PE Applied Biosystems, Foster City, Calif.) was combined with 5 ng of PCR product obtained by the method of Example 1 which served as the template. To this was added 3.2 pmol of primers and deionized water to 10 μl. Primers used were the same as those used in the original PCR amplification. The cycling protocol consisted of 25 cycles of a 10 second denaturation step at 96° C., a 5 second annealing step at 50° C. and a 4 minute extension step at 60° C. After the last cycle, the reaction mixture was cooled to 4° C. until purification. Unincorporated dye was removed from the sequencing products by ethanol precipitation and loaded onto sequencing cells on either Applied Biosystems (ABI 377) or Licor automatic gel sequencers. Two μl samples in sample buffer (5:1 100% formamide:blue dextran dye) were loaded onto sequencing gels and run at 2.4 kV for 6 hours in 1×TBE running buffer. Laser scans of the gel were at a rate of 1200 per hour. Peaks generated were analyzed by eye for heterozygosity. On sample was run per lane of the gel.

Results

A deletion polymorphism was found at position 1272 of SEQ ID NO: 1 in which the reference sequence C at position 1272 is deleted. This mutation was found in 27% of patients with ESRD due to NIDDM and 20% of patients with ESRD due to HTN, but not in the reference sequence.

This deletion causes disruption of a potential NF-1 (nuclear factor 1) site (CTTTGGCACTACCCAAAA) (SEQ ID NO: 20) beginning at position 1259 on the (−) strand. NF-1 sites occur relatively frequently with 4.11 sites per 1000 base pairs of random genomic DNA in vertebrates. Since NF-1 is a transcriptional activator, disruption of its binding site is expected to result in a decreased rate of transcription of the ecNOS gene. If the rate of translation is tied to the level of messenger RNA, as is the case for most proteins, then less gene product (ecNOS enzyme) will be the result, ultimately leading to less nitric oxide (NO) produced in tissues such as endothelial cells.

This deletion also causes disruption of a potential BARBIE (barbiturate-inducible element) site (TGCCAAAGCGTAAGG) (SEQ ID NO: 21) beginning at position 1269 on the (+) strand. BARBIE is a transcriptional regulator not yet linked with regulation of the ecNOS gene. BARBIE sites occur with considerably less frequency than NF-1 sites at a rate of 0.56 times per 1000 base pairs of random genomic sequence in vertebrates.

### Example 5

### T to A Substitution at Position 2841

DNA isolation, purification, amplification and sequencing were as described in Example 4 except the forward primer was 5′ gagtctggccaacacaaatcc 3′ (SEQ ID NO: 3) and the reverse primer was 5′ctctagggtcatgcaggttctc 3′ (SEQ ID NO: 22).

A substitution polymorphism (transversion) was found in which the reference sequence T at position 2841 of SEQ ID NO: 1 is replaced with an A. This polymorphism was found in 29% of patients with ESRD due to NIDDM, but not in the reference sequence or patients with ESRD due to HTN.

This polymorphism disrupts the predicted binding site of NFY (nuclear factor Y), with sequence GCCCCAATTTC, (SEQ ID NO: 23) ending at position 2837 on the (−) strand. The T2837→A polymorphism replaces the nucleotide T on the (+) strand with an A. The corresponding reference sequence nucleotide on the (−) strand is therefore changed from the A, indicated in the NFY binding site sequence immediately above, to a T. Disruption of the NFY binding site is expected to result in reduced transcription of the ecNOS gene, since NFY is a potent transcriptional activator. NFY binding sites occur with extreme rarity, <0.01 sites per 1000 base pairs of random genomic sequence in vertebrates. Thus, finding a SNP at this site is strongly suggestive that it is a causal SNP in end-stage renal disease due to NIDDM.

### Example 6

### G to T Substitution at Position 2843

DNA isolation, purification, amplification and sequencing were as described in Example 5.

A substitution polymorphism (transversion) was found in which the reference sequence G at position 2843 of SEQ ID NO: 1 is replaced with a T. This polymorphism was found in 29% of patients with ESRD due to NIDDM and 14% of patients with ESRD due to HTN, but not in the reference sequence.

This polymorphism disrupts the predicted binding site of NFY (nuclear factor Y), GCCCCAATTTC, (SEQ ID NO: 23) ending at position 2837 of SEQ ID NO: 1 on the (−) strand. The G-630→T polymorphism replaces the reference sequence nucleotide G on the (+) strand with a T. The corresponding nucleotide on the (−) strand is therefore changed from the C, indicated in the NFY binding site sequence immediately above, to an A. Disruption of the NFY binding site in this core region is expected to result in reduced transcription of the ecNOS gene, since NFY is a potent transcriptional activator. NFY binding sites occur with extreme rarity, <0.01 sites per 1000 base pairs of random genomic sequence in vertebrates. Thus, finding a SNP at this site is strongly suggestive that it is a causal SNP in end-stage renal disease due to NIDDM, and, to a lesser extent, hypertension.

### Example 7

### G to T Substitution at Position 3556

DNA isolation, purification, amplification and sequencing were as described in Example 4 except that the forward primer was 5′ atccttgctgggcctctat 3′ (SEQ ID NO: 24) and the reverse primer was 5′tgcttgccgcacagcccaa3′ (SEQ ID NO: 25).

A substitution polymorphism (transversion) was found in which the G at position 3556 of SEQ ID NO: 1 is replaced with a T. This polymorphism was found in 50% of patients with ESRD due to HTN, but not in the reference sequence or patients with ESRD due to NIDDM.

This polymorphism produces a missense mutation of Glycine in exon 1 (encoded by GGG, codon 18) to Tryptophan (encoded by TGG). This G18W amino acid mutation replaces a small amino acid with a bulky hydrophobic one, which may interfere with protein conformation and ultimately enzymatic activity. Reduced enzymatic activity would result in decreased nitric oxide production in tissues, consistent with the results predicted for all of the above SNPS.

### CONCLUSION

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventor does not intend to be bound by those conclusions and functions, but puts them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

What is claimed is:

1. A method for diagnosing a genetic predisposition for a disease, condition or disorder in a subject comprising, obtaining a biological sample containing nucleic acid from said subject; and analyzing said nucleic acid to detect the presence or absence of a single nucleotide polymorphism in SEQ ID NO: 1 or the complement thereof, wherein said single nucleotide polymorphism is associated with a genetic predisposition for a disease selected from the group consisting of hypertension, end stage renal disease due to hypertension, non-insulin dependent diabetes mellitus, and end stage renal disease due to non-insulin dependent diabetes mellitus.

2. The method of claim 1, wherein said nucleic acid is DNA, cDNA, RNA, or mRNA.

3. The method of claim 1, wherein said single nucleotide polymorphism is located at position 2548 or 2684 of SEQ ID NO: 1.

4 The method of claim 3, wherein said single nucleotide polymorphism is selected from the group consisting of G2548→A, C2548′→-T, C2684→T, and G2684′→A.

5. The method of claim 1, wherein said analysis is accomplished by sequencing, mini sequencing, hybridization, restriction fragment analysis, oligonucleotide ligation assay, or allele specific PCR.

6. A method for diagnosing a genetic predisposition for a disease, condition or disorder in a subject comprising, obtaining a biological sample containing nucleic acid from said subject; and analyzing said nucleic acid to detect the presence or absence of a single nucleotide polymorphism in SEQ ID NO: 1 or the complement thereof, wherein said single nucleotide polymorphism is associated with a genetic predisposition for a disease selected from the group consisting of hypertension, end stage renal disease due to hypertension, non-insulin dependent diabetes mellitus, end stage renal disease due to non-insulin dependent diabetes mellitus, breast cancer, lung cancer and prostate cancer.

7. The method of claim 6, wherein said nucleic acid is DNA, RNA, cDNA or mRNA.

8. The method of claim 6, wherein said single nucleotide polymorphism is located at position 2548, 2684, 2575, 1272, 2841, 2843 or 3556 of SEQ ID NO: 1.

9. The method of claim 6, wherein said single nucleotide polymorphism is selected from the group consisting of G2548→A, C2684→T, C2575→T, C1272 deletion, T2841→A, G2843→T, G3556→T, C2548′→T C2684′→A, G2575′→A, G1272′ deletion, A2841′→T, and C2843′→A.

10. An isolated polynucleotide comprising at least 10 contiguous nucleotides of SEQ ID NO: 1 or the complement thereof, and containing at least one single nucleotide polymorphism at position 2548, 2575, 1272, 2841, 2843 or 3556 of SEQ ID NO: 1, wherein said at least one single nucleotide polymorphism is associated with a disease selected from the group consisting of hypertension, end stage renal disease due to hypertension, non-insulin dependent diabetes mellitus, end stage renal disease due to non-insulin dependent diabetes mellitus, breast cancer, lung cancer and prostate cancer.

11. The isolated polynucleotide of claim 10, wherein said at least one single nucleotide polymorphism is selected from the group consisting of G2548→A, C2575→T, C1272 deletion, T2841→A, G2843→T, G3556→T, C2548→T, G2575′→A, G1272′ deletion, A2841′→T, and C2843′→A.

12. The isolated polynucleotide of claim 10, wherein said single nucleotide polymorphism is located at the 3′ end of said polynucleotide.

13. The isolated polynucleotide of claim 10, further comprising a detectable label.

14. The isolated polynucleotide of claim 13, wherein said detectable label is selected from the group consisting of radionuclides, fluorophores or fluorochromes, peptides, enzymes, antigens, antibodies, vitamins and steroids.

15. A kit comprising at least one isolated polynucleotide of at least 10 continuous nucleotides of SEQ ID NO: 1 or the complement thereof, and containing at least one single nucleotide polymorphism associated with a disease, condition or disorder selected from the group consisting of hypertension, end stage renal disease due to hypertension, non-insulin dependent diabetes mellitus, end stage renal disease due to non-insulin dependent diabetes mellitus, breast cancer, lung cancer and prostate cancer; and instructions for using said polynucleotide for detecting the presence or absence of said single nucleotide polymorphism in said nucleic acid.

16. The kit of claim 15, wherein said single nucleotide polymorphism is located at position 2548, 2684, 2575, 1272, 2841, 2843 or 3556 of SEQ ID NO: 1.

17. The kit of claim 16, wherein said single nucleotide polymorphism is selected from the group consisting of G2548→A, C2684→T, C2575→T, C1272 deletion, T2841+A, G2843→T, G3556→T, C2548→T C2684′→A, G2575→A, G1272′ deletion, A2841′→T, and C2843′-+A.

18. The kit of claim 15, wherein said single nucleotide polymorphism is located at the 3′ end of said polynucleotide.

19. The kit of claim 15, wherein said polynucleotide further comprises at least one detectable label.

20. The kit of claim 19, wherein said label is selected from the group consisting of radionuclides, flurorphores or fluorochromes, peptides, enzymes, antigens, antibodies, vitamins or steroids

21. A kit comprising at least one polynucleotide of at least 10 contiguous nucleotides of SEQ ID NO: 1 or the complement thereof, wherein the 3′ end of said polynucleotide is immediately 5′ to a single nucleotide polymorphism site associated with a genetic predisposition to disease condition, or disorder selected from the group consisting of hypertension, end stage renal disease due to hypertension, non-insulin dependent diabetes mellitus, end stage renal disease due to non-insulin dependent diabetes mellitus, breast cancer, lung cancer and prostate cancer; and instructions for using said polynucleotide for detecting the presence or absence of said single nucleotide polymorphism in a biological sample containing nucleic acid.

22. The kit of claim 21, wherein said single nucleotide polymorphism site is located at position 2548, 2684, 2575, 1272, 2841, 2843 or 3556 of SEQ ID NO: 1.

23. The kit of claim 21, wherein said polynucleotide further comprises a detectable label.

24. The kit of claim 23, wherein said detectable label is selected from the group consisting of radionuclides, fluororphores or fluorochromes, peptides, enzymes, antigens antibodies, vitamins and steroids.

25. A method for treatment or prophylaxis in a subject comprising, obtaining a sample of biological material containing nucleic acid from a subject; analyzing said nucleic acid to detect the presence or absence of at least one single nucleotide polymorphism in SEQ ID NO: 1 or the complement thereof associated with a disease, condition or disorder selected from the group consisting of hypertension, end stage renal disease due to hypertension, non-insulin dependent diabetes mellitus, end stage renal disease due to non-insulin dependent diabetes mellitus, breast cancer, lung cancer and prostate cancer; and treating said subject for said disease, condition or disorder.

26. The method of claim 25 wherein said nucleic acid is selected from the group consisting of DNA, cDNA, RNA and mRNA.

27. The method of claim 25 wherein said single nucleotide polymorphism is located at position 2548, 2684, 2575, 1272, 2841, 2843 or 3556 of SEQ ID NO: 1.

28. The method of claim 27 wherein said single nucleotide polymorphism is selected from the group consisting of G2548→A, C2684→T, C2575→T, C1272 deletion, T2841→A, G2843→T, G3556→T, C2548′→T C2684′→A, G2575′→A, G1272′ deletion, A2841′→T, and C2843′→A.

29. The method of claim 25 wherein said treatment increases the production of nitric oxide.

30. The method of claim 29 wherein said treatment comprises administration of L-arginine.

31. The method of claim 25 wherein said treatment counteracts the effect of said at least one single nucleotide polymorphism detected.


SEQUENCE LISTING (25 sequences)

SEQ ID NO: 1 - DNA, 3586 bp, Homo sapiens
  misc_feature (1465)..(3585): Promotor region and exon 1, partial CDS
gggcccagagaaagagctgtccccggggccttggggacagggtgacagccacccagagatcatggagaaggggacgtaaggaagacctcacagaggagtcatcctgcgactgtgttggttgggtccttcaggaagcagagtcccaggagttggaagcataagaggaatactgcgggcaatgcctgagaaagataacagggaccgggagcaggagtgagttgggcaggggaaggatcaggcccacaatgccaggctcacacctgcagaggagggaagaagaagaagggcctcacatcagcccagcgggggatgttacgcccacagacgccccggggctcagttactgtctaagtgttagaaataaattttcggtgccacaaaagaaatagcactcagattaaatgttcccagcaaggcaattttacttctatagaagggtgcatctcacagatggagcaatggcaagagcacacctgaacaagggaagggaaggggtttttatccctaaggcaggtagcccctacagctgtgttgttcccctattggctagggttggaccacaccgtctgagctaattgttactggctattttaaagagagcaggggtaagagccggattggcagggtaagtagtttggcaggaaggacggtcacagaacaggtgactcaggatgactcaggtcagagcaggtgaccagtggtgactcagttcggagcaggtgatagaagctaggagggggttgtttactgaaactaggggcaaggagacgaagagaacatgaaagttaaactttaagatgaagaacaaagctgaacatactgatgcattggatctttggagaggatctcagaactcattgtacttaatttacaggctaaaaccttagaagaggaatttattatatcctacacaagactccagggaagcacatggccttggactgaaggctggcatctggaagctgtcagccaccagcaccttctgcagcaggtacctgctctctaagagggaggcctgggtggtgcacctccagagctgcccaggctgggcctcaaggaagaaaaagattttcatttgtcagaggcggaagggagaggtggagggaacagcacagcagcggcccaggggcagggaagcacaggaccattagggagacacgagaaagcccatttgtctagaacagaggattcaagcagtgcaccaaggaaaatgagggccaggccaatgtgctggagtggctttgttcttggctgagggttttgggtagtgccaaagcgtaaggtaagccctgctttccagaagaatctagcagagtgtggagcccagatgggactggaaggcctgggaggggtcaggtggccacagggacgggccacagccagtggtgcaggcaagaagacaatggccatccatggtggctcacacctggaatcccagcccattgggaggtcgaggcaggtggatcacctgaggtcaggagttcgagaccagcctggtcaacatggtgaaaccctgtctctaataaaattataaaaattagccgggcgtggtggtgggtacctgtaatctcagctactcaggaggctgggtcaggagaatcgcttgaacccaggaggcggaggttacagtgagctgagatagcaccattgcattccagcctggacaacaaaagcgagactctgtctcaaaaaaaaaaaaaaattagccaggcgtggtggtgggtgcctgtcgtcctcgggaggctgaggcatgagaatcactccgggaggcagaggttgcaatgaaccaagatcacaccactgcactccagcctgggtgacagagcaagactctgtctaaaaaaaaaaaaaagacagaaggatgtcagcatctgatgctgcctgtcaccttgaccctgaggatgccagtcacagctccattaactgggacctaggaaaatgagtcatccttggtcatgcacatttcaaatggtggcttaatatggaagccacacttgggatctgttgtctcctccagcatggtagaagatgcctgaaaagtaggggctggatcccatcccctgcctcactgggaaggcgaggtggtggggtggggtggggcctcaggcttggggtcatgggacaaagcccaggctgaatgccgcccttccatctccctcctcctgagacaggggcagcagggcacactagtgtccaggagcagcttatgaggccccttcaccctccgatcctccaaaactggcagaccccaccttcttcggtgtgaccccagagctctgagcacagcccgttccttccgcctgccggccccccacccaggcccaccccaaccttatcctccactgcttttcagaggagtctggccaacacaaatcctcttgtttgtttgtctgtctgtctgctgctcctagtctctgcctctcccagtctctcagcttccgtttctttcttaaactttctctcagtctctgaggtctcgaaatcacgaggcttcgacccctgtggaccagatgcccagctagtggcctttctccagcccctcagatggcacagaactacaaaccccagcatgcactctggcctgaagtgcctggagagtgctggtgtaccccacctgcattctgggaactgtagtttccctagtcccccatgctcccaccagggcatcaagctcttccctggccggctgaccctgcctcagccctagtctctctgctgacctgcggccccgggaagcgtgcgtcactgaatgacagggtgggggtggaggcactggaaggcagcttcctgctcttttgtgtcccccacttgagtcatgggggtgtgggggttccaggaaattggggctgggaggggaagggataccctaatgtcagactcaaggacaaaaagtcactacatccttgctgggcctctatccccaagaacccaaaaggactcaagggtggggatccaggagttcttgtatgtatggggggaggtgaaggagagaacctgcatgaccctagaggtccctgtggtcactgagagtgtgggctgccatcccctgctacagaaacggtgctcaccttctgcccaaccctccagggaaaggcacacaggggtgaggccgaaccttccgtctggtgccacatcacagaaggacctttatgaccccctggtggctctaccctgccactccccaatgccccagcccccatgctgcagccccagggctctgctggacacctgggctcccacttatcagcctcagtcctcacagcggaacccaggcgtccggccccccacccttcaggccagcgggcgtggagctgaggctttagagcctcccagccgggcttgttcctgtcccattgtgtatgggataggggcggggcgagggccagcactggagagccccctcccactgccccctcctctcggtcccctccctcttcctaaggaaaaggccagggctctgctggagcaggcagcagagtggacgcacagtaacatgggcaacttgaagagcgtggcccaggagcctgggccaccctgcggcctggggctggggctgggccttgggctgtgcggcaagcagggcccn

SEQ ID NO: 2 - protein, 31 aa, Homo sapiens
  MISC_FEATURE (1)..(31): Promotor region and exon 1, partial CDS
MGNLKSVAQEPGPPCGLGLGLGLGLCGKQGP

SEQ ID NO: 3 - DNA, 21 bp, Artificial sequence
  Forward Primer
gagtctggccaacacaaatcc

SEQ ID NO: 4 - DNA, 22 bp, Artificial sequence
  Reverse Primer
ctctagggtcatgcaggttctc

SEQ ID NO: 5 - DNA, 18 bp, Homo sapiens
  misc_feature (1)..(18): NF-1 (nuclear factor 1) site
agatggcacagaactaca

SEQ ID NO: 6 - DNA, 10 bp, Homo sapiens
  misc_feature (1)..(10): Myoblast determining factor binding site
gccatctgag

SEQ ID NO: 7 - DNA, 12 bp, Homo sapiens
  Misc_feature (1)..(12): LMO2COM (complex of Lmo2 bound to Tal-1, E2A protein) binding site
cctcagatggca

SEQ ID NO: 8 - DNA, 16 bp, Homo sapiens
  misc_feature (1)..(16): TAL1ALPHAE47 (Tal-1alpha/E47 heterodimer) binding site, TAL1BETAE 47 (Tal-1beta/E47 heterodimer) binding site
cccctcagatggcaca

SEQ ID NO: 9 - DNA, 18 bp, Homo sapiens
  misc_feature (1)..(18): NF1 (nuclear factor 1) binding site/Estrogen receptor binding site
ccctggccggctgaccct

SEQ ID NO: 10 - DNA, 13 bp, Homo sapiens
  misc_feature (1)..(13): TCF11 (TCF11/KCR-F1/Nrf1 homodimer) binding site
gtcagccggccag

SEQ ID NO: 11 - DNA, 10 bp, Homo sapiens
  misc_feature (1)..(10): AP4 (activator protein 4) binding site
gtcagccggc

SEQ ID NO: 12 - DNA, 19 bp, Homo sapiens
  misc_feature (1)..(19): VMAF (v-Maf) binding site
gccggctgaccctgcctca

SEQ ID NO: 13 - DNA, 21 bp, Artificial sequence
  Forward Primer
gagtctggccaacacaaatcc

SEQ ID NO: 14 - DNA, 22 bp, Artificial sequence
  Reverse Primer
ctctagggtcatgcaggttctc

SEQ ID NO: 15 - DNA, 22 bp, Homo sapiens
  Misc_feature (1)..(22): STAF_1 (Se-Cys tRNA gene transcription activating factor)
aaaccccagcatgcactctggc

SEQ ID NO: 16 - DNA, 16 bp, Homo sapiens
  Misc_feature (1)..(16): TH1E47_01 (Thing1/E47 heterodimer) site
catgcactctggcctg

SEQ ID NO: 17 - DNA, 18 bp, Homo sapiens
  Misc_feature (1)..(18): NF1_Q6 (nuclear factor 1) site
ctctggcctgaagtgcct

SEQ ID NO: 18 - DNA, 24 bp, Artificial sequence
  Forward Primer
agcagtgcaccaaggaaaatgagg

SEQ ID NO: 19 - DNA, 24 bp, Artificial sequence
  Reverse Primer
agtgcagtggtgtgatcttggttc

SEQ ID NO: 20 - DNA, 18 bp, Homo sapiens
  Misc_feature (1)..(18): Potential NF-1 (nuclear factor 1) site
ctttggcactacccaaaa

SEQ ID NO: 21 - DNA, 15 bp, Homo sapiens
  Misc_feature (1)..(15): BARBIE (barbiturate-inducible element) site
tgccaaagcgtaagg

SEQ ID NO: 22 - DNA, 22 bp, Artificial sequence
  Reverse Primer
ctctagggtcatgcaggttctc

SEQ ID NO: 23 - DNA, 11 bp, Homo sapiens
  Misc_feature (1)..(11): NFY (nuclear factor Y)
gccccaatttc

SEQ ID NO: 24 - DNA, 19 bp, Artificial sequence
  Forward Primer
atccttgctgggcctctat

SEQ ID NO: 25 - DNA, 19 bp, Artificial sequence
  Reverse Primer
tgcttgccgcacagcccaa